SEQUENCE LISTING (47 sequences)

SEQ ID NO: 1 - DNA, 45 bp, synthetic construct
  misc_feature 1..45: note = CD64 leader signal sequence
  source 1..45: mol_type = other DNA
atgtggtttcttactacattgctgttgtgggtcccggtggacggt

SEQ ID NO: 2 - DNA, 321 bp, synthetic construct
  misc_feature 1..321: note = FMC63 light chain variable region
  source 1..321: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 3 - DNA, 75 bp, synthetic construct
  misc_feature 1..75: note = G-S spacer
  source 1..75: mol_type = other DNA
tcctcagggggcgggggaagtggtgggggcggcagcggcggagggggctcaggaggaggcggatcaggcggatca

SEQ ID NO: 4 - DNA, 360 bp, synthetic construct
  misc_feature 1..360: note = FMC63 heavy chain variable region
  source 1..360: mol_type = other DNA
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 5 - DNA, 663 bp, synthetic construct
  misc_feature 1..663: note = Ectodomain, transmembrane and signaling domains
  source 1..663: mol_type = other DNA
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 6 - protein, 267 aa, synthetic construct
  REGION 1..267: note = CD19-SPScFvFMC63
MWFLTTLLLWVPVDGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSSGGGGSGGGGSGGGGSGGGGSGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 7 - DNA, 327 bp, synthetic construct
  misc_feature 1..327: note = SHIE2 light chain variable region
  source 1..327: mol_type = other DNA
cagtctgccctgactcagcctgcctccgtgtctgggtctcttggacagtcagtcaccatctcctgcactggaagcagcagtgatgttgggagttataaccttgtctcctggtaccaacagcacccaggcaaagcccccaatctcatgatttatgatgtcagtaagcggtcaggggtttctaatcgcttctctggctccaagtctggcaacacggcctccctgaccatctctgggctccaggctgaggacgaggctgattattactgcagctcatatacaggcatcagcaccgtggtattcggcggagggaccaagctgaccgtcctg

SEQ ID NO: 8 - DNA, 60 bp, synthetic construct
  misc_feature 1..60: note = linker
  source 1..60: mol_type = other DNA
ggcggcggaggaagcggaggcggaggatctgggggcggaggctctggcggagggggatct

SEQ ID NO: 9 - DNA, 357 bp, synthetic construct
  misc_feature 1..357: note = SHIE2 heavy chain variable region
  source 1..357: mol_type = other DNA
gaggtgcagctggtggagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagccccgtactactactactacatggacgtctggggccaagggaccacggtcaccgtgagctca

SEQ ID NO: 10 - DNA, 327 bp, synthetic construct
  misc_feature 1..327: note = RBSC6 light chain variable region
  source 1..327: mol_type = other DNA
aacatccagatgacccagtctccatcttctgtgtctgcatctgtaggagacagagtcaccatcacttgtcgggcgagtcaggatattagccgctggttagcctggtatcagcagaaaccagggaaagcccctaaactcctgatctatgctgcatccagtttgcaaagtggggtcccatcgaggttcagcggcagtggatctgggacagatttcgctctcactatcagcagcctgcagcctgaagattttgcaacttactattgtcaacaggctgacagtcgtttctcgatcaccttcggccaagggacacgactggagattaaaggc

SEQ ID NO: 11 - DNA, 57 bp, synthetic construct
  misc_feature 1..57: note = G-S linker
  source 1..57: mol_type = other DNA
ggcggaggaagcggaggcggaggatctgggggcggaggctctggcggagggggatct

SEQ ID NO: 12 - DNA, 354 bp, synthetic construct
  misc_feature 1..354: note = RBSC6 heavy chain variable region
  source 1..354: mol_type = other DNA
gaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatacagtacgcagactctgtgaagggccgattcaccatctccagagacaatgccaagaactcactgtatctgcaaatgaacagcctgagagacgaggacacggctgtgtattactgtgcgagaggggactactactacggtatggacgtctggggccaagggaccacggtcaccgtgagctca

SEQ ID NO: 13 - DNA, 336 bp, synthetic construct
  misc_feature 1..336: note = Ganitumab light chain variable region
  source 1..336: mol_type = other DNA
gatgttgtaatgacgcagtcacccctgtcactcccggtcacacccggagaaccagcgtcaattagctgccgatctagccaaagtttgcttcattccaatggttacaattatctcgactggtacttgcagaaacccggccaatcccctcagctgctcatctaccttgggtctaatagggcatctggggttcccgataggttctctggctccgggagcggcaccgactttacgttgaaaatctctagggttgaggcggaagacgtaggcgtttactattgcatgcaggggacccactggccgctgaccttcggccagggcaccaaggttgaaataaaa

SEQ ID NO: 14 - DNA, 366 bp, synthetic construct
  misc_feature 1..366: note = Ganitumab heavy chain variable region
  source 1..366: mol_type = other DNA
ggcggatcacaggtacagctccaggaatcaggacccggtttggttaagccctccgggaccctttccctcacgtgtgcagtctcaggtgggtcaattagttcttccaattggtggtcttgggtgcggcaaccacctggtaaaggtctcgagtggataggggaaatttatcatagtggctccaccaattataacccctcactcaagtccagggttacgatatctgtggacaaaagtaaaaaccaattctccctcaaacttagtagtgtaacagcggcagacaccgcggtgtactactgcgcacggtggacaggccgaactgatgcctttgacatttggggacagggaactatggtgactgtgtcatcc

SEQ ID NO: 15 - DNA, 321 bp, synthetic construct
  misc_feature 1..321: note = Cetuximab light chain variable region
  source 1..321: mol_type = other DNA
gatattcttcttactcaatctcccgttattttgtcagtatccccaggtgagcgagtcagcttctcttgtcgagcgtcacaatccattggcaccaacatacattggtaccaacagcgcaccaacgggtctccccggctcttgattaagtacgcatcagaaagtatttctgggatacccagtaggttctcagggagcgggagtggcactgactttaccctgtccataaacagcgttgagtctgaggacatcgcggactactattgtcagcagaacaacaattggccgaccacgtttggtgcgggaacaaaacttgaactcaaa

SEQ ID NO: 16 - DNA, 357 bp, synthetic construct
  misc_feature 1..357: note = Cetuximab heavy chain variable region
  source 1..357: mol_type = other DNA
caggtgcagctcaaacagtcaggacctggcctcgttcagccaagccaatcactgagtataacgtgcacggtgagcggctttagcctgacaaactatggtgtccactgggtccgccaatctcctggaaaaggcttggagtggctcggtgttatctggtccggtggtaacacagactacaacacgccattcaccagtcgccttagtattaacaaggacaactccaagtctcaggttttctttaaaatgaactctctgcagtctaatgataccgcaatttactactgtgcgagggcactcacgtactatgactatgagttcgcgtattggggccaagggactctcgttactgtctcagcg

SEQ ID NO: 17 - DNA, 364 bp, synthetic construct
  misc_feature 1..364: note = FRP5 heavy chain variable region
  source 1..364: mol_type = other DNA
ggacggtcaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctct

SEQ ID NO: 18 - DNA, 318 bp, synthetic construct
  misc_feature 1..318: note = FRP5 light chain variable region
  source 1..318: mol_type = other DNA
gacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatc

SEQ ID NO: 19 - DNA, 328 bp, synthetic construct
  misc_feature 1..328: note = Trastuzumab heavy chain variable region
  source 1..328: mol_type = other DNA
ggacggtgacatccagatgacccagagccccagcagcctgagcgccagcgtgggcgacagagtgaccatcacctgcagagccagccaggacgtgaacaccgccgtggcctggtaccagcagaagcccggcaaggcccccaagctgctgatctacagcgccagcttcctgtacagcggcgtgcccagcagattcagcggcagcagaagcggcaccgacttcaccctgaccatcagcagcctgcagcccgaggacttcgccacctactactgccagcagcactacaccaccccccccaccttcggccagggcaccaaggtggagatcaag

SEQ ID NO: 20 - DNA, 360 bp, synthetic construct
  misc_feature 1..360: note = Trastuzumab light chain variable region
  source 1..360: mol_type = other DNA
gaggtgcagctggtggagagcggcggcggcctggtgcagcccggcggcagcctgagactgagctgcgccgccagcggcttcaacatcaaggacacctacatccactgggtgagacaggcccccggcaagggcctggagtgggtggccagaatctaccccaccaacggctacaccagatacgccgacagcgtgaagggcagattcaccatcagcgccgacaccagcaagaacaccgcctacctgcagatgaacagcctgagagccgaggacaccgccgtgtactactgcagcagatggggcggcgacggcttctacgccatggactactggggccagggcaccctggtgaccgtgagcagc

SEQ ID NO: 21 - protein, 220 aa, synthetic construct
  REGION 1..220: note = CAR-CD19-CD28/CD3zeta (CAR-19slh28zeta)
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - DNA, 453 bp, synthetic construct
  misc_feature 1..453: note = CD3zeta
  source 1..453: mol_type = other DNA
cagctgcccatcaccgaggctcaatcctttgggctgcttgatcctaagttgtgttatcttcttgacggcatcctgtttatatacggtgtcattttgacagctctctttctccgagtcaagttcagtcggtctgcagatgctccagcataccagcaaggccaaaatcagctgtataacgagttgaaccttgggcgacgagaagaatacgacgtgcttgacaaacgcagaggtcgggaccctgaaatggggggtaagccgcaaaggcggaagaatccacaagagggtctgtataatgaattgcaaaaagacaagatggctgaggcatactcagagattggaatgaaaggggaaagacggaggggaaagggccacgatggactgtaccaagggctgagcaccgcaactaaagatacatatgatgctctgcacatgcaggccctgccacctagataa

SEQ ID NO: 23 - protein, 150 aa, synthetic construct
  REGION 1..150: note = CD3zeta
QLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - DNA, 48 bp, synthetic construct
  misc_feature 1..48: note = Ectodomain
  source 1..48: mol_type = other DNA
cagctgcccatcaccgaggctcaatcctttgggctgcttgatcctaag

SEQ ID NO: 25 - DNA, 64 bp, synthetic construct
  misc_feature 1..64: note = Transmembrane domain
  source 1..64: mol_type = other DNA
ttgtgttatcttcttgacggcatcctgtttatatacggtgtcattttgacagctctctttctcc

SEQ ID NO: 26 - DNA, 341 bp, synthetic construct
  misc_feature 1..341: note = Cytoplasmic domain
  source 1..341: mol_type = other DNA
gagtcaagttcagtcggtctgcagatgctccagcataccagcaaggccaaaatcagctgtataacgagttgaaccttgggcgacgagaagaatacgacgtgcttgacaaacgcagaggtcgggaccctgaaatggggggtaagccgcaaaggcggaagaatccacaagagggtctgtataatgaattgcaaaaagacaagatggctgaggcatactcagagattggaatgaaaggggaaagacggaggggaaagggccacgatggactgtaccaagggctgagcaccgcaactaaagatacatatgatgctctgcacatgcaggccctgccacctagataa

SEQ ID NO: 27 - DNA, 117 bp, synthetic construct
  misc_feature 1..117: note = Ectodomain
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 28 - DNA, 78 bp, synthetic construct
  misc_feature 1..78: note = Transmembrane domain
  source 1..78: mol_type = other DNA
ttttgggtgctggtggtggttggtggagtcctggcttgctatagcttgctagtaacagtggcctttattattttctgg

SEQ ID NO: 29 - DNA, 468 bp, synthetic construct
  misc_feature 1..468: note = Cytoplasmic domain
  source 1..468: mol_type = other DNA
gtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 30 - DNA, 66 bp, synthetic construct
  misc_feature 1..66: note = G-S linker
  source 1..66: mol_type = other DNA
tcctcagggggcgggggaagtggtgggggcggcagcggcggagggggctcaggaggaggcggatca

SEQ ID NO: 31 - DNA, 1247 bp, synthetic construct
  misc_feature 1..1247: note = CD64-VH domain-GS spacer-VL domain-CD3zeta complete
  source 1..1247: mol_type = other DNA
atgtggtttcttactacattgctgttgtgggtcccggtggacggtcaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctaaaagtgaagacagtgctacatatttctgtgcaagatgggaggtttaccacggctacgttccttactggggccaagggaccacggtcaccgtttcctcttcctcagggggcgggggaagtggtgggggcggcagcggcggagggggctcaggaggaggcggatcaggcggatcagacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatccagctgcccatcaccgaggctcaatcctttgggctgcttgatcctaagttgtgttatcttcttgacggcatcctgtttatatacggtgtcattttgacagctctctttctccgagtcaagttcagtcggtctgcagatgctccagcataccagcaaggccaaaatcagctgtataacgagttgaaccttgggcgacgagaagaatacgacgtgcttgacaaacgcagaggtcgggaccctgaaatggggggtaagccgcaaaggcggaagaatccacaagagggtctgtataatgaattgcaaaaagacaagatggctgaggcatactcagagattggaatgaaaggggaaagacggaggggaaagggccacgatggactgtaccaagggctgagcaccgcaactaaagatacatatgatgctctgcacatgcaggccctgccacctagataa

SEQ ID NO: 32 - protein, 415 aa, synthetic construct
  REGION 1..415: note = CD64-VH domain-GS spacer-VL domain-CD3zeta complete
MWFLTTLLLWVPVDGQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSSSGGGGSGGGGSGGGGSGGGGSGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - protein, 109 aa, synthetic construct
  REGION 1..109: note = SHIE2 light chain variable region
QSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVL

SEQ ID NO: 34 - protein, 20 aa, synthetic construct
  REGION 1..20: note = linker
GGGGSGGGGSGGGGSGGGGS

SEQ ID NO: 35 - protein, 119 aa, synthetic construct
  REGION 1..119: note = SHIE2 heavy chain variable region
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSS

SEQ ID NO: 36 - protein, 109 aa, synthetic construct
  REGION 1..109: note = RBSC6 light chain variable region
NIQMTQSPSSVSASVGDRVTITCRASQDISRWLAWYQQKPGKAPKLLIYAASSLQSGVPSRFSGSGSGTDFALTISSLQPEDFATYYCQQADSRFSITFGQGTRLEIKG

SEQ ID NO: 37 - protein, 118 aa, synthetic construct
  REGION 1..118: note = RBSC6 heavy chain variable region
EVQLVQSGGGLVQPGGSLRLSCAASGFTFSSYSMNWVRQAPGKGLEWVSYISSSSSTIQYADSVKGRFTISRDNAKNSLYLQMNSLRDEDTAVYYCARGDYYYGMDVWGQGTTVTVSS

SEQ ID NO: 38 - protein, 15 aa, synthetic construct
  REGION 1..15: note = CD64 leader signal sequence
MWFLTTLLLWVPVDG

SEQ ID NO: 39 - protein, 25 aa, synthetic construct
  REGION 1..25: note = G-S spacer
SSGGGGSGGGGSGGGGSGGGGSGGS

SEQ ID NO: 40 - protein, 112 aa, synthetic construct [sequence omitted]

SEQ ID NO: 41 - protein, 119 aa, synthetic construct [sequence omitted]

SEQ ID NO: 42 - protein, 107 aa, synthetic construct
  REGION 1..107: note = Cetuximab light chain variable region
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 43 - protein, 121 aa, synthetic construct
  REGION 1..121: note = Cetuximab heavy chain variable region
QVQLKQSGPGLVQPSQSLSITCTVSGFSLTNYGVHWVRQSPGKGLEWLGVIWSGGNTDYNTPFTSRLSINKDNSKSQVFFKMETNSLQSNDTAIYYCARALTYYDYEFAYWGQGTLVTVSA

SEQ ID NO: 44 - protein, 125 aa, synthetic construct
  REGION 1..125: note = FRP5 heavy chain variable region
QVQLQQSGPELKKPGETVKISCKASGYPFTNYGMETNWVKQAPGQGLKWMETGWINTSTGESTFADDFKGRFDFSLETSANTAYLQINNLKSEDMETATYFCARWEVYHGYVPYWGQGTTVTVSS

SEQ ID NO: 45 - protein, 106 aa, synthetic construct
  REGION 1..106: note = FRP5 light chain variable region
DIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEI

SEQ ID NO: 46 - protein, 109 aa, synthetic construct
  REGION 1..109: note = Trastuzumab heavy chain variable region
DIQMETTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 47 - protein, 124 aa, synthetic construct
  REGION 1..124: note = Trastuzumab light chain variable region
EVQLVESGGGLVQPGGSLRLSCAASGFNIKDTYIHWVRQAPGKGLEWVARIYPTNGYTRYADSVKGRFTISADTSKNTAYLQMETNSLRAEDTAVYYCSRWGGDGFYAMETDYWGQGTLVTVSS

(19) United States
(12) Patent Application Publication
(10) Pub. No.: US 2025/0206800 A1
(43) Pub. Date: Jun. 26, 2025

(54) Chimeric T Cell Receptors, Nucleic Acids, And Methods Of Making And Using The Same

(71) Applicant: NantBio, Inc., Culver City, CA (US)

(72) Inventor: Hermes GARBÁN, Los Angeles, CA (US)

(21) Appl. No.: 19/053,508

(22) Filed: Feb. 14, 2025

Related U.S. Application Data
(63) Continuation of application No. 17/387,347, filed on Jul. 28, 2021, now Pat. No. 12,252,525.
(60) Provisional application No. 63/059,560, filed on Jul. 31, 2020.

(51) Int. Cl. C07K 14/725; A61K 40/15; A61K 40/31; A61K 40/42; A61K 40/50; C07K 14/705

(52) U.S. Cl. CPC C07K 14/7051; A61K 40/15; A61K 40/31; A61K 40/42; C07K 14/70521; A61K 40/50; C07K 2317/622; C07K 2319/02; C07K 2319/03

(57) ABSTRACT

Compositions and methods for eradicating tumor cells using novel compositions are contemplated. In one aspect, a pharmaceutical composition comprising a CAR scaffold and an antigen binding domain in a single chimeric species is provided. In some aspects, the CAR scaffold may comprise a CD28 costimulatory signaling region and a CD3ζ (activation domain or a complete CD3ζ activation domain. In some aspects, the CAR scaffold may be codon-optimized for improved expression in mammalian cell lines and/or for improved function upon transfection into natural killer (NK) or other immune cells. In further aspects, the antigen binding domain may comprise a VL and VH domain linked by a spacer and may be codon optimized. A CD64 leader sequence may be attached to the antigen binding domain, e.g., at the N-terminus of the antigen binding domain.

### CROSS-REFERENCE TO RELATED PATENT APPLICATIONS

This patent application is a continuation of U.S. patent application Ser. No. 17/387,347 filed 28 Jul. 2021, which claims the benefit under 35 U.S.C. § 119 (e) of U.S. Provisional Patent Application No. 63/059,560 filed on 31 Jul. 2020. The entire contents of each application recited above is incorporated by reference.

### FIELD

The field of the invention relates to chimeric receptor genes that encode chimeric T cell receptors (TCRs), which provide antibody-type specificity to immune cells that are transformed with said genes, and methods of using the same, for the treatment of diseases such as cancer.

### BACKGROUND

The background description includes information that may be useful in understanding the compositions and methods described herein. It is not an admission that any of the information provided herein is prior art or relevant to the compositions and methods, or that any publication specifically or implicitly referenced is prior art.

In recent years, immunotherapy has emerged as a therapeutic option for the treatment of cancer. A variety of monoclonal antibodies have been approved by regulatory agencies and are approved for use in the treatment of various types of cancers. Additional approaches are under investigation including cancer vaccines and adjuvants, chimeric antigen receptors (CAR) T-cell therapies, immune checkpoint inhibitors, and combinations thereof.

However, there is still a need to provide new compositions and methods that specifically target tumor cells for treatment, including solid and blood-based malignancies. Accordingly, there is still a need to provide improved compositions and methods of CAR T-cells with improved properties over their predecessors.

### SUMMARY

The methods and techniques provided herein are directed to various systems and methods of developing and administering targeted immune therapy, as well as nucleic acids encoding the same.

In one aspect, a pharmaceutical composition comprising a CAR scaffold and an antigen binding domain in a single chimeric species is provided. In some aspects, the CAR scaffold may comprise a CD28 costimulatory signaling region and a CD3ζ activation domain. The CD28 costimulatory signaling region may comprise an ectodomain, a transmembrane domain, and a cytoplasmic domain. In some aspects, the cytoplasmic domain may include a CD28 co-activation domain. In other aspects, the CD3ζ activation domain may comprise at least two immunoreceptor tyrosine-based activation motifs (ITAMs). In some aspects, the CAR scaffold may be codon-optimized for improved expression in mammalian cell lines and/or for improved function upon transfection into natural killer (NK) or other types of immune cells. In further aspects, the antigen binding domain may comprise a VL and VH domain linked by a spacer. A CD64 leader sequence may be attached to the antigen binding domain, e.g., at the N-terminus of the antigen binding domain.

The compositions provided herein include chimeric TCRs and nucleic acids encoding chimeric TCRs. Methods of making and of administering the chimeric TCRs are also provided.

The compositions provided herein may be provided to any suitable cell, including NK cells, helper T cells, suppressor T cells, cytotoxic T cells, lymphokine-activated T cells, and any other immune cell capable of expressing a chimeric receptor. Suitable NK cells include but are not limited to high-affinity natural killer cells (haNKs), tumor activated natural killer cells (taNKs), and activated natural killer cells (aNKs).

In some aspects, the antigen binding domain may comprise an scFv, which includes a VL domain connected to a VH domain by a spacer. The scFv may include VH and VL domains from any suitable monoclonal antibody, including but not limited to a monoclonal antibody that has been approved by or is under investigation by a regulatory agency, e.g., cetuximab, ganitumab, etc.

In other aspects, the antigen binding domain and scaffold may be provided without a signaling domain, such that contact between the CAR scaffold expressing NK cell and the antigen expressing cell stimulates lysis of the antigen expressing cell.

In other aspects, the antigen binding domain may comprise an antibody fragment, including but not limited to a scFv, a di-scFv, a Fab, a Fab′, a F(ab′)2, a sdAb, or complementary determining regions (CDRs). In still other aspects, the antigen binding domain may comprise an aptamer, an affimer, a peptide, a protein, a protein scaffold, a small molecule, etc.

In still other aspects, the antigen binding domain may comprise a protein binding domain, including but not limited to FK506-binding protein 5 (FKBP5). Any suitable binding protein, which binds to an antigen on the surface of a target cell, are suitable for use with the CAR scaffolds provided herein.

The antibody fragment may be human, humanized, synthetic, or chimeric.

In other aspects, the present subject matter may also include DNA vectors comprising the nucleic acids disclosed herein, and host cells for expressing the DNA vectors. Nucleotides encoding the antigen binding domain and CAR scaffold may be inserted into any suitable expression vector, examples of which are known in the art. Host cells may include but are not limited to, a mammalian cell, an insect cell, a eukaryotic cell, a prokaryotic cell, a yeast cell, an animal cell, or a human cell. The host cell may also include, but is not limited to, a CHO cell, a NSO cell, a SP2-O cell, or a J558 cell.

Further contemplated are methods of making and expressing constructs comprising the antigen binding domain and CAR scaffold. In some aspects, the method includes: (a) introducing into a host cell a DNA encoding an antigen binding domain coupled to a CAR scaffold; and (b) culturing the host cell in media under conditions sufficient to express the antigen binding domain and CAR scaffold.

According to present techniques, a method for treating cancer in a subject is contemplated. The method may comprise administering to said subject a therapeutically effective amount of a pharmaceutical composition comprising the antigen binding domain coupled to a CAR scaffold. As used herein, “therapeutically effective amount” refers to administration of a pharmaceutical composition according to a dosage amount and/or a dosage regimen sufficient to treat a specific disease. One of ordinary skill in the art will appreciate that a therapeutically effective amount or dose may depend on a variety of factors, including the type of tumor or cancer, the route of administration of the therapeutic composition, the characteristics of the patient, the extent to which the tumor or cancer has metastasized, and/or the overall health of the patient. Effective doses may also be extrapolated from dose-response curves derived from in vitro or animal model test systems.

In some embodiments, the cancer is selected from but not limited to the group consisting of brain, breast, cervical, colon, gastric, lung, beta-cell lymphoma, melanoma, multiple myeloma, mesothelioma, ovarian, prostate, pancreatic, renal, thyroid, urothelial/bladder carcinoma, and hematological malignancies, e.g., acute myelogenous leukemia (AML), chronic lymphocytic leukemia (CLL), and Hodgkin's lymphoma. The techniques presented herein are applicable to any type of tumor, including but not limited to, primary tumors, secondary tumors, recurrent tumors, and derivative tumors.

In some aspects, administration occurs once, twice, or three times per week. As used herein, “administration” refers to the administration of the pharmaceutical composition to a patient in need thereof. In other aspects, the pharmaceutical composition may be administered by any suitable route, including but not limited to, bronchial, enteral, interdermal, intra-arterial, intradermal, intragastric, intramedullary, intramuscular, intranasal, intrathecal, intravenous, intraventricular, mucosal, nasal, oral, rectal, subcutaneous, sublingual, topical, tracheal, transdermal, vaginal, and vitrael.

Various uses of the aforementioned compositions are also contemplated herein. Various embodiments of the aforementioned compositions may also be administered to a patient in need thereof. Various objects, features, aspects and advantages of the compositions will become more apparent from the following detailed description of preferred embodiments.

### BRIEF DESCRIPTION OF THE DRAWINGS

FIG. 1 is an illustration showing various domains of the antigen binding domain and CAR scaffold, according to examples provided herein. The antigen binding domain includes a CD64 leader sequence attached to the N-terminal of a scFv binding molecule, wherein the VL and VH domains are connected to each other via a spacer. The scFv domain is coupled to a CAR scaffold, comprising a CD28 domain and an activation domain. The CD28 domain comprises an extracellular domain, a transmembrane domain, a CD28 co-activation domain, and is coupled to a CD3ζ activation domain comprising at least two ITAM regions.

FIGS. 2A-2B are illustrations showing alternative configurations of CAR-T receptors, for comparison to the CAR scaffolds provided herein. FIG. 2A shows a CAR construct comprising a single chain antibody fragment coupled to an extracellular CD8 hinge region. The hinge region is connected to a CD28 transmembrane domain that is linked to an intracellular signaling domain, in this case, a FcεRIγ signaling domain (instead of a CD28-CD3ζ construct as shown in FIG. 1). This CAR construct has shown cytotoxicity/ADCC in comparative studies.

FIG. 2B shows an illustration of a tricistronic construct, which may be inserted into a vector, such as a pNEUKv1 vector. This construct comprises a nucleotide sequence encoding (in order) a CD19CAR domain, a P2A sequence, an optional linker, a CD16 (158V) domain, an IRES sequence, and an ERIL-2 domain. The tricistronic construct incorporates expression of a high affinity variant of the Fc receptor (CD16-158V) and allows growth activity in the absence of exogenous IL-2.

FIGS. 3A-3B show additional illustrations of a CD28-CD3ζ construct according to aspects provided herein. FIG. 3A shows a detailed functional organization of an antigen binding domain coupled to a CD28-CD3ζ scaffold. The antigen binding domain comprises a FMC63-V_{L} domain—a GS spacer—a FMC63-V_{H} domain (also referred to as 19slh28ζ). The antigen binding domain and CAR scaffold may be inserted into any suitable vector, and may comprise about 1464 bp. In this figure, the locations of the CDRs (CDR1, CDR2, CDR3 for the heavy and light chains are shown).

FIG. 3B shows another illustration of the 19slh28ζ (p123) construct, which is similar to FIG. 1 and includes FMC63-VL and FMC63-VH domains, linked by a SGₓ spacer. The various domains of the CAR scaffold, including the CD28 and CD3ζ activation domains, are shown. Control vectors (not shown) may be used for functional comparison as described herein.

FIG. 4 shows nucleotide sequences for a human codon optimized DNA sequence (CAR19slh28ζ (αCD19-CD28/CD3ζ)). Nucleotide sequences encoding various domains are shown, including a nucleotide sequence that encodes a CD64 leader sequence (SEQ ID NO.: 1), nucleotide sequences that encode variable regions (SEQ ID NOs.: 2,4), a nucleotide sequence that encodes a G-S spacer (SEQ ID NO.: 3), and a nucleotide sequence that encodes ectodomain, transmembrane and signaling domains (SEQ ID NO.: 5). As shown previously, SEQ ID NOs.: 1-5 may be linked together to form a full-length CAR construct that encodes the antigen binding domain.

SEQ ID NO.: 5 represents the CAR scaffold for FIGS. 4-17, unless indicated otherwise. Further, FIGS. 4-17 correspond to nucleotide sequences that have been codon optimized for expression in human cell lines, in some cases, resulting in an improvement in expression levels of the CAR scaffold construct up to 25% above a non-codon optimized construct. Thus, one or more of the nucleotide sequences encoding the VH, VL and/or CD28/CD3ζ domains may be codon optimized.

FIG. 5 shows another representation of the nucleotide sequence of FIG. 4 but limited to the sequences encoding the antigen binding domain. Various domains are shown, including a nucleotide sequence that encodes a CD64 leader sequence (SEQ ID NO.: 1), nucleotide sequences that encode variable regions (SEQ ID NOs.: 2, 4), and a nucleotide sequence that encodes a G-S spacer (SEQ ID NO.: 3). The sequences are linked together (leader—variable region—spacer—variable region) to form, upon expression, a scFv sequence. These sequences have been codon optimized for expression in human cells.

FIG. 6 shows an amino acid sequence (SEQ ID NO.: 6), corresponding to the nucleotide sequence of (FIG. 5) for a FMC63-derived binding domain in a scFv format. The first underlined portion is the CD64 leader sequence, followed by a variable region (FMC63-VL), a second underlined portion representing a G-S spacer, followed by a variable region (FMC63-VH).

FIGS. 7A-7B show experimental data comparing specific cell lysis amounts of the constructs in FIGS. 1 and 2B. These constructs represent anti-CD19 CARs that bind to CD19, a B-lineage specific transmembrane glycoprotein expressed in more than 95% of B-cell malignancies. As this protein is a cell-surface protein, it is an ideal target for novel CAR based immunotherapies. These anti-CD19 CARs may target cytotoxic cells to the malignant cells.

FIG. 7A shows specific lysis percentages based on a ratio of effector to target cells in sup-B15 cells. Both the anti-CD19 scFv tricistronic construct and the anti-CD19 scFv CD28CD3ζ construct showed higher cell lysis percentages than the control. The tricistronic construct appeared to have somewhat higher cell lysis percentages than the CD28-CD3ζ construct. These differences may be attributed in part due to expression levels of the antigen binding domain, which may vary based on cell type. FIG. 7B shows specific lysis percentages based on a ratio of effector to target cells in Ramos cells. Both the anti-CD19 scFv tricistronic construct and the anti-CD19 scFv CD28-CD3ζ construct showed higher cell lysis percentages than the control. The tricistronic construct and the CD28-CD3ζ construct showed comparable (within 10%) lysis percentages.

FIG. 8A shows an illustration of an anti-αPD-L1 (SHIE2) scFv construct. Various domains are shown, including a CD64 leader domain, heavy and light chain variable regions, as well as a linker.

FIG. 8B shows the corresponding nucleotide sequences encoding the domains of FIG. 8A, including nucleotide sequences that encode a leader sequence (SEQ ID NO.: 1), anti-αPD-L1 light and heavy chains (SEQ ID NOs.: 7, 9), and a linker (SEQ ID NO.: 8). This nucleotide sequence (leader—variable region—linker—variable region) has been codon optimized for expression in human cells.

FIG. 9A shows an illustration of an anti-αPD-L1 (RBSC6) scFv construct that is human codon optimized. Various domains are shown, including a CD64 leader sequence and heavy and light chain variable regions (RBSC6-VL and RBSC6-VH) connected by a linker/spacer. The variable regions are linked together to form a scFv sequence.

FIG. 9B shows nucleotide sequences encoding the anti-αPD-L1 (RBSC6) scFv construct of FIG. 9A, including nucleotide encoding a CD64 leader sequence (SEQ ID NO.: 1), a variable region (RBSC6-VL) (SEQ ID NO.: 10), a linker (SEQ ID NO.: 11), and another variable region (RBSC6-VH) (SEQ ID NO.: 12). This nucleotide sequence (leader—variable region—spacer—variable region) has been codon optimized for expression in human cells.

FIG. 10 shows experimental data comparing specific cell lysis percentages of the expressed constructs of FIGS. 8A-8B and 9A-9B. These constructs represent anti-PD-L1 CARs. PD-L1, a transmembrane glycoprotein, is expressed in different types of cancer cells and is an ideal target for CAR-based immunotherapies. Anti-PD-L1 antibodies target PD-L1 on the surface of cancer cells to trigger cell killing of the malignant cells. FIG. 10 shows specific lysis percentages of the anti-PD-L1 CARs based on a ratio of effector to target cells in MDA-MB-231cells. Both the RBSC6 scFv tricistronic construct and the SH1E2 scFv tricistronic construct showed higher cell lysis percentages than the control. Both the SH1E2 scFv CD28-CD3ζ construct and the RBSC6 scFv CD28-CD3ζ construct showed a higher cell lysis percentages than the tricistronic constructs.

FIG. 11A shows an illustration of an αIGF1R (ganitumab) scFv construct that is human codon optimized. Various domains are shown, including a CD64 leader sequence and heavy and light chain variable regions (Gani-VL and Gani-VH) connected by a G-S spacer. The variable regions are linked together via the spacer to form a scFv sequence.

FIG. 11B shows the corresponding nucleotide sequences of the domains of the construct of FIG. 11A. Nucleotide sequences encoding the leader sequence (SEQ ID NO.: 1), ganitumab light chain (SEQ ID NO.: 13), linker (SEQ ID NO.: 30), and ganitumab heavy chain (SEQ ID NO.: 14) are shown. This sequence (leader—variable region—spacer—variable region) has been codon optimized for expression in human cells.

FIG. 12A shows an illustration of an αEGFR (cetuximab) scFv construct. Various domains are shown, including a CD64 leader sequence and heavy and light chain variable regions connected by a G-S spacer that to form a scFv sequence.

FIG. 12B shows the corresponding nucleotide sequences of the domains of the αEGFR scFv construct of FIG. 12A. Nucleotide sequences encoding the leader (SEQ ID NO.: 1), anti-αEGFR light chain (SEQ ID NO.: 15), G-S spacer (SEQ ID NO.: 3), and anti-αEGFR heavy chain (SEQ ID NO.: 16) are shown. This sequence (leader—variable region—spacer—variable region) has been codon optimized for expression in human cells.

FIGS. 13A-13B show experimental data comparing specific cell lysis percentages of the constructs of FIGS. 11A-11B and 12A-12B. These constructs represent anti-IGFR-1 or anti-EGFR CARs. IGFR-1 and EGFR are transmembrane glycoproteins, expressed in different types of cancer cells, and are ideal targets for CAR-based immunotherapies. FIG. 13A shows specific lysis percentages based on a ratio of effector to target cells in A549 cells. Both the anti-EGFR scFv tricistronic construct and the anti-EGFR scFv CD28/CD3ζ construct showed higher cell lysis percentages than the control. The anti-EGFR scFv CD28-CD3ζ construct showed generally higher lysis percentages than the anti-EGFR tricistronic construct.

FIG. 13B shows specific lysis percentages based on a ratio of effector to target cells in MDA-MB-231 cells. Both the anti-IGFR-1 scFv CD28-CD3ζ construct and the anti-IGFR-1 scFv tricistronic construct showed higher cell lysis percentages than the control. The anti-IGFR-1 scFv CD28-CD3ζ construct showed somewhat higher lysis percentages than the anti-IGFR-1 scFv tricistronic construct.

FIG. 14A shows an illustration of a FRP5 scFv construct that is human codon optimized. Various domains are shown, including a CD64 leader sequence and heavy and light chain variable regions connected by a G-S spacer/linker. The variable regions are linked together to form a scFv sequence. The antigen binding domain may bind to HER2.

FIG. 14B shows the corresponding nucleotide sequences of the domains of the αERBB2 (FRP5) scFv construct of FIG. 14A. Nucleotide sequences encoding the leader (SEQ ID NO.: 1), (αFRP5-VH) FRP5 heavy chain (SEQ ID NO.: 17), linker (SEQ ID NO.: 3), and (αFRP5-VL) FRP5 light chain (SEQ ID NO.: 18) are shown. This sequence (leader—variable region—spacer—variable region) has been optimized for expression in human cells.

FIG. 15A shows an illustration of a trastuzumab scFv construct that is human codon optimized. Various domains are shown, including a CD64 leader sequence and heavy and light chain variable regions connected by a G-S spacer to form a scFv sequence.

FIG. 15B shows the corresponding nucleotide sequences of the domains of the construct of FIG. 15A. Nucleotide sequences encoding the CD64 leader (SEQ ID NO.: 1), trastuzumab-VH (SEQ ID NO.: 19), G-S spacer (SEQ ID NO.: 3), and trastuzumab-VL (SEQ ID NO.: 20) are provided. This sequence (leader—variable region—spacer—variable region) has been optimized for expression in human cells.

FIG. 16 shows experimental data comparing specific cell lysis percentages of the constructs of FIGS. 14A-14B and 15A-15B. The FRP5 CARS and trastuzumab CARs both bind to HER2(αERBB2). FIG. 16 shows specific lysis percentages based on a ratio of effector to target cells in SK-OV3 cells. Both the αERBB2/FRP5 scFv tricistronic construct and the αERBB2/FRP5 scFv CD28-CD3ζ construct showed higher cell lysis percentages than the control.

FIG. 17 shows nucleotide sequences encoding a CAR scaffold (SEQ ID NO.: 5) comprising a CD28 co-stimulatory segment from CD28 and an activation domain of CD3ζ with the two final immunoreceptor tyrosine-based activation motif (ITAMs)). The antigen binding domain is followed by the CD28-derived hinge (ectodomain), transmembrane and coactivating cytoplasmic domains followed by a distal activating domain from CD3ζ. Nucleotide sequence (SEQ ID NO.: 5) may be translated to generate amino acid sequence (SEQ ID NO.: 21).

FIGS. 18A-18B show a schematic representation of another CAR construct containing a CD3ζCO activation domain. This construct contains the complete CD3ζ gene, including an ectodomain (hinge), a transmembrane domain, and a cytoplasmic domain (e.g., including 3×ITAMS). This construct has been codon optimized (CO).

FIG. 18C shows a nucleotide sequence that has been codon optimized for human expression, corresponding to the domains of FIGS. 18A-18B. Codon optimization improves expression (e.g., higher levels of the construct), which may result in improved efficacy (killing) once transfected into NK cells (e.g., taNKs)). Nucleotide sequence (SEQ ID NO.: 22) may be translated to generate amino acid sequence (SEQ ID NO.: 23).

FIGS. 18D and 18E show codon optimized nucleotides encoding the scFv-CD3ζ (SEQ ID NO.: 22) and scFv-CD28/CD3ζ (SEQ ID NO.: 5) scaffolds. FIG. 18D shows nucleotides encoding the ectodomain (SEQ ID NO.: 24), transmembrane domain (SEQ ID NO.: 25), and cytoplasmic domain (SEQ ID NO.: 26) for the scFv-CD3ζ domain (SEQ ID NO.: 22). FIG. 18E shows nucleotide sequences encoding the ectodomain (SEQ ID NO.: 27), transmembrane domain (SEQ ID NO.: 28), and cytoplasmic domain (SEQ ID NO.: 29) for the scFv-CD28/CD3ζ domain (SEQ ID NO.: 5).

FIG. 19 shows multiple schematic representations of constructs according to the techniques provided herein. The activation domain comprises a transmembrane component and a CD3ζ with three ITAMs, and may be codon optimized (CO) (P-WT-0031 and P-WT-0032) or may not be codon optimized (P-WT-0029 and P-WT-0030). In some cases, a CD28 hinge domain may be present (P-WT-0029 and P-WT-0031). Heavy and light chain domains may be obtained from any suitable monoclonal antibody (e.g., cetuximab, ganitumab, etc.). Any suitable expression vector may be used, e.g., pRNi, pShuttle-CMV, etc. Constructs may include single vector constructs such as scFvs. Alternatively, constructs may include pShuttle-CMV vectors that allow for expression of HC and LC chains separately and formation of the antigen binding domain in vivo.

The P-WT-0029 and 0030 scaffolds include native T cell structures. P-WT-0031 corresponds to a scaffold comprising a CD28 hinge+CD3ζ, and P-WT-0032 corresponds to a full CD3ζ scaffold. Any of these scaffolds may be incorporated into NK cells.

FIG. 20A shows a schematic illustration of the construction, orientation, and restriction sites a construct according to present techniques. Restriction sites are shown, along with relevant domains (CD64-VH domain-G-S spacer/linker-VL domain-CD3ζ complete CO).

FIG. 20B shows a sequence alignment of the nucleotide sequence (SEQ ID NO: 31), amino acid sequence (SEQ ID NO: 32), CDRs, and domains of the construct illustrated in FIG. 20.

FIG. 21A shows the construction, orientation, and restriction sites of SPScFvFM63-VLVH-28Z 19 slh285. FIG. 21B shows the sequence alignment, restriction sites, nucleotide sequence, and amino acid sequence of SPScFvFM63-VLVH-28Z 19 slh28ζ. The nucleotide sequence corresponds to, sequentially, SEQ ID NOs: 1-5 corresponding to CD64 signal sequence, FMC63-VL (also described as FMC63 light chain variable region), G-S spacer, FMC63-VH (also described as FMC63 heavy chain variable region), and collectively CD28 and CD3ζ activation domain (also described as the ectodomain, transmembrane, and signaling domains), respectively. The amino acid sequence corresponds to SEQ ID NO: 6, corresponding to CD19-SPScFvFMC63, and SEQ ID NO: 21, corresponding to CAR-CD19-CD28/CD3.

FIG. 22A shows the construction and orientation of CD19-SPScFvFMC63, corresponding to a CD64 signal sequence, FMC63-VL, G-S spacer, and FMC63-VH, respectively. FIG. 22B shows the amino acid sequence of CD19-SPScFvFMC63 (SEQ ID NO: 6).

FIG. 23A shows the construction, orientation, and restriction sites of αPD-L1 SHIE2 pNBS-XL52 ScFv. FIG. 23B shows the sequence alignment, restriction sites, nucleotide sequence, and amino acid sequence of αPD-L1 SHIE2 pNBS-XL52 ScFv. The nucleotide sequence corresponds to, sequentially, SEQ ID NOs: 1, 7, 8, and 9, corresponding to CD64 signal sequence, SHIE2 VL (also described as SHIE2 light chain variable region), linker, and SHIE2 VH (also described as SHIE2 heavy chain variable region), respectively. The amino acid sequence corresponds to, sequentially, SEQ ID NOs: 38 and 33-35 corresponding to CD64, SHIE2 VL, linker, and SHIE2 VH, respectively.

FIG. 24A shows the construction, orientation, and restriction sites of αPD-L1 (RBSC6) ScFv, corresponding to a CD64 signal sequence, RBSC6 VL, linker, and RBSC6 VH, respectively. FIG. 24B shows the sequence alignment, restriction sites, nucleotide sequence, and amino acid sequence of αPD-L1 (RBSC6) ScFv. The nucleotide sequence corresponds to, sequentially, SEQ ID NOs: 1, 10, 8, and 12, corresponding to CD64 signal sequence, RBSC6 VL (also described as RBSC6 light chain variable region), linker, and RBSC6 VH (also described as RBSC6 heavy chain variable region), respectively. The amino acid sequence corresponds to, sequentially, SEQ ID NOs: 38, 36, 34, and 37 corresponding to CD64, RBSC6 VL, linker, and RBSC6 VH, respectively.

FIG. 25A shows the construction, orientation, and restriction sites of αIGF1R (Ganitumab) ScFv, corresponding to a CD64 signal sequence, Gani VL, G-S spacer, and Gani VH, respectively. FIG. 25B shows the sequence alignment, restriction sites, nucleotide sequence, and amino acid sequence of αIGF1R (Ganitumab) ScFv. The nucleotide sequence corresponds to, sequentially, SEQ ID NOs: 1, 13, 3, and 14, corresponding to CD64 signal sequence, Gani VL (also described as Ganitumab light chain variable region), G-S spacer, and Gani VH (also described as Ganitumab heavy chain variable region), respectively. The amino acid sequence corresponds to, sequentially, SEQ ID NOs: 38, 40, 39, and 41 corresponding to CD64, Gani VL, G-S spacer, and Gani VH, respectively.

FIG. 26A shows the construction, orientation, and restriction sites of αEGFR (Cetuximab) pXL101b, corresponding to a CD64 signal sequence, Cetuximab VL, G-S spacer, and Cetuximab VH, respectively. FIG. 26B shows the sequence alignment, restriction sites, nucleotide sequence, and amino acid sequence of αEGFR (Cetuximab) pXL101b. The nucleotide sequence corresponds to, sequentially, SEQ ID NOs: 1, 15, 3, and 16, corresponding to CD64 signal sequence, Cetuximab VL (also described as Cetuximab light chain variable region), G-S spacer, and Cetuximab VH (also described as Ganitumab heavy chain variable region), respectively. The amino acid sequence corresponds to, sequentially, SEQ ID NOs: 38, 42, 39, and 43 corresponding to CD64, Cetuximab VL, G-S spacer, and Cetuximab VH, respectively.

FIG. 27A shows the construction, orientation, and restriction sites of αERBB2 (FRP5) ScFv pXL011, corresponding to a CD64 signal sequence, FRP5 VH, G-S linker, and FRP5 VL, respectively. FIG. 27B shows the sequence alignment, restriction sites, nucleotide sequence, and amino acid sequence of αERBB2 (FRP5) ScFv pXL011. The nucleotide sequence corresponds to, sequentially, SEQ ID NOs: 1, 17, 3, and 18, corresponding to CD64 signal sequence, FRP5 VH (also described as FRP5 heavy chain variable region), G-S linker, and FRP5 VL (also described as FRP5 light chain variable region), respectively. The amino acid sequence corresponds to, sequentially, SEQ ID NOs: 38, 44, 39, and 45 corresponding to CD64, FRP5 VL, G-S linker, and FRP5 VH, respectively.

FIG. 28A shows the construction, orientation, and restriction sites of αERBB2 (Trastuzumab) ScFv TSHL28z-p147, corresponding to a CD64 signal sequence, Trastuzumab VH, G-S spacer, and Trastuzumab VL, respectively. FIG. 28B shows the sequence alignment, restriction sites, nucleotide sequence, and amino acid sequence of αERBB2 (FRP5) ScFv pXL011. The nucleotide sequence corresponds to, sequentially, SEQ ID NOs: 1, 19, 3, and 20, corresponding to CD64 signal sequence, Trastuzumab VH (also described as Trastuzumab heavy chain variable region), G-S spacer, and Trastuzumab VL (also described as Trastuzumab light chain variable region), respectively. The amino acid sequence corresponds to, sequentially, SEQ ID NOs: 38, 46, 39, and 47 corresponding to CD64, Trastuzumab VL, G-S spacer, and Trastuzumab VH, respectively.

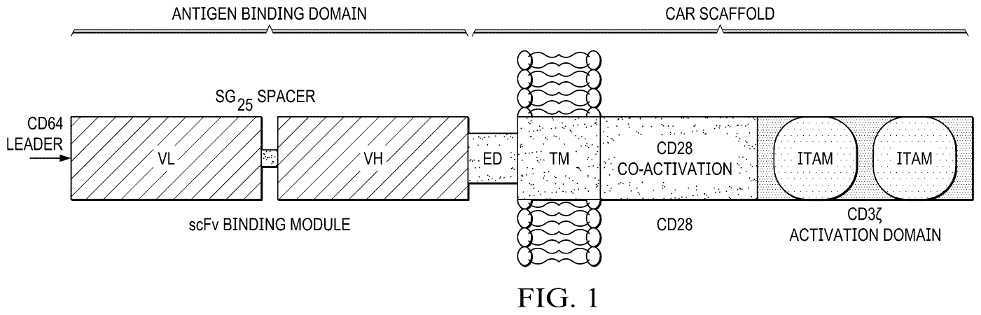

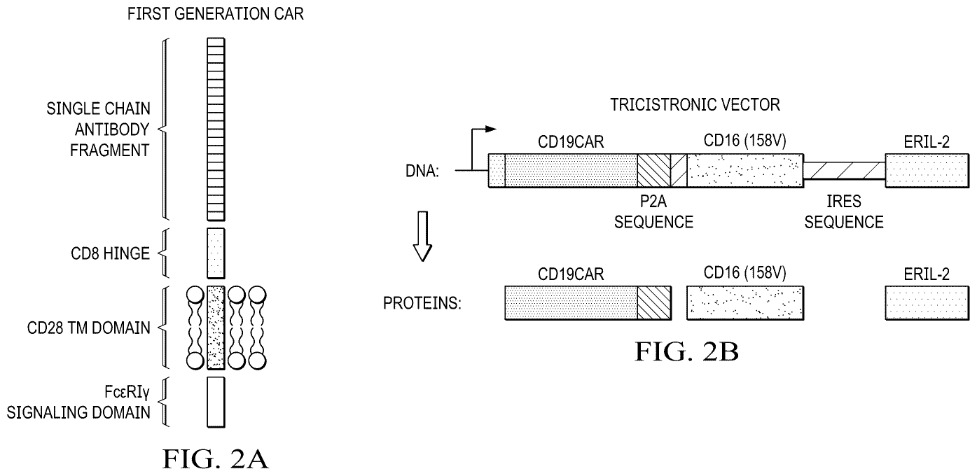

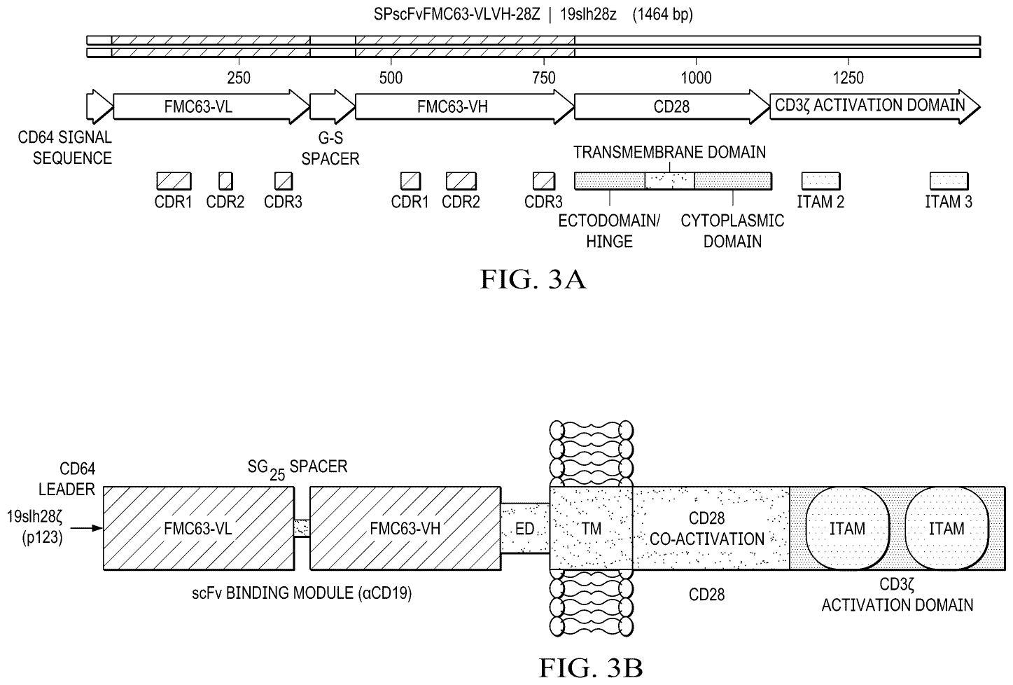

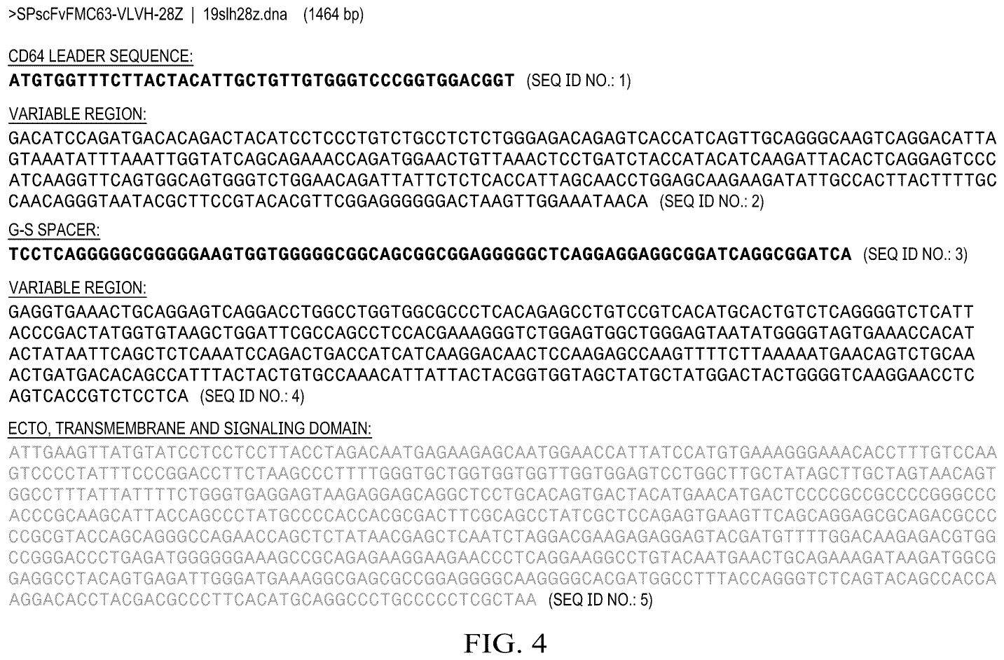

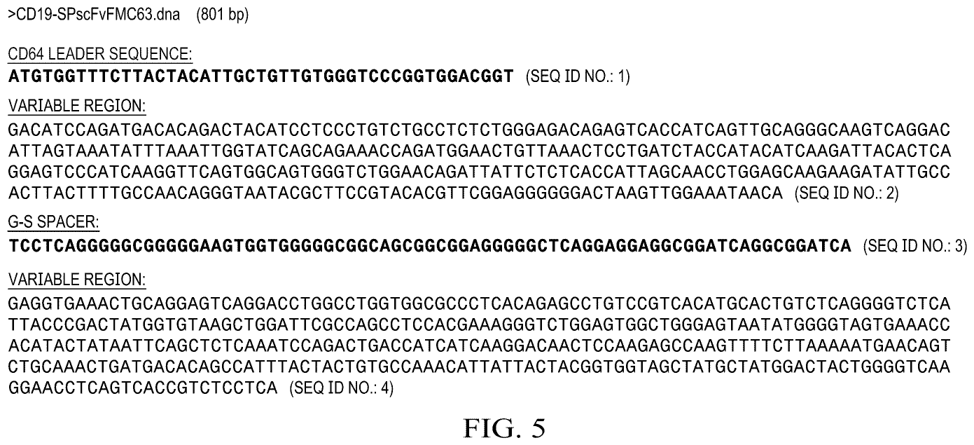

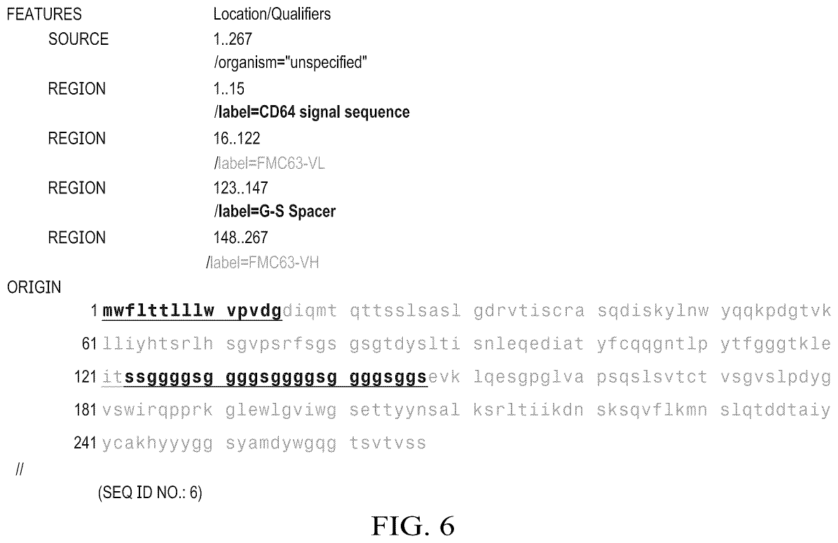

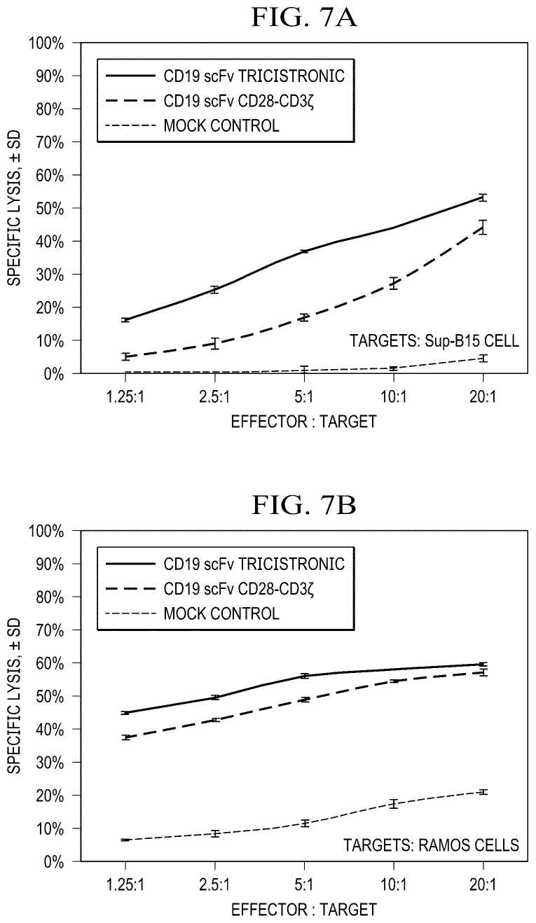

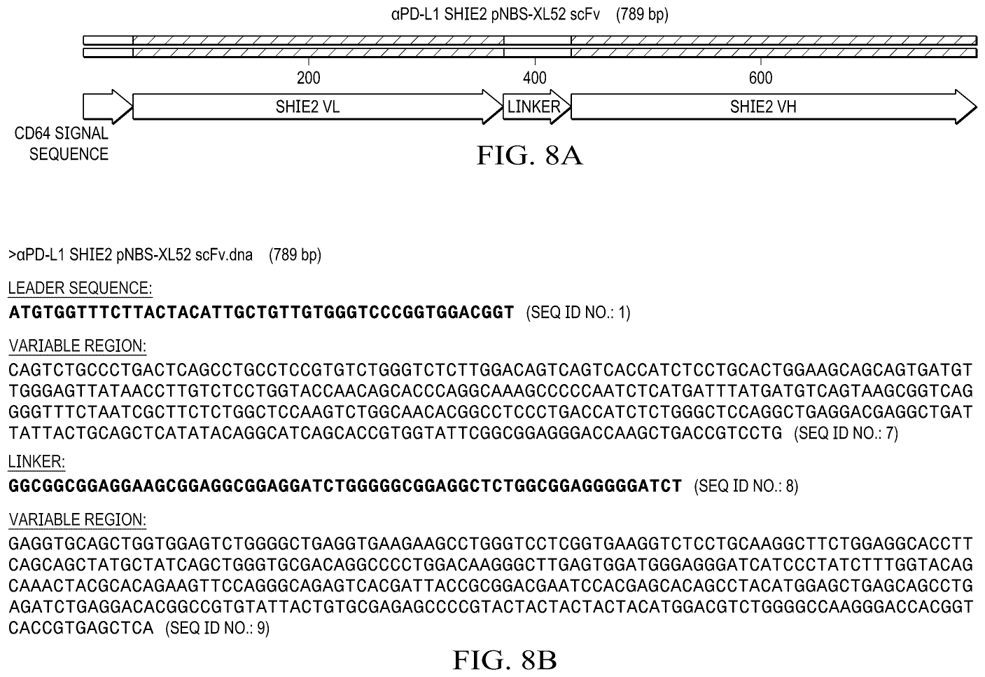

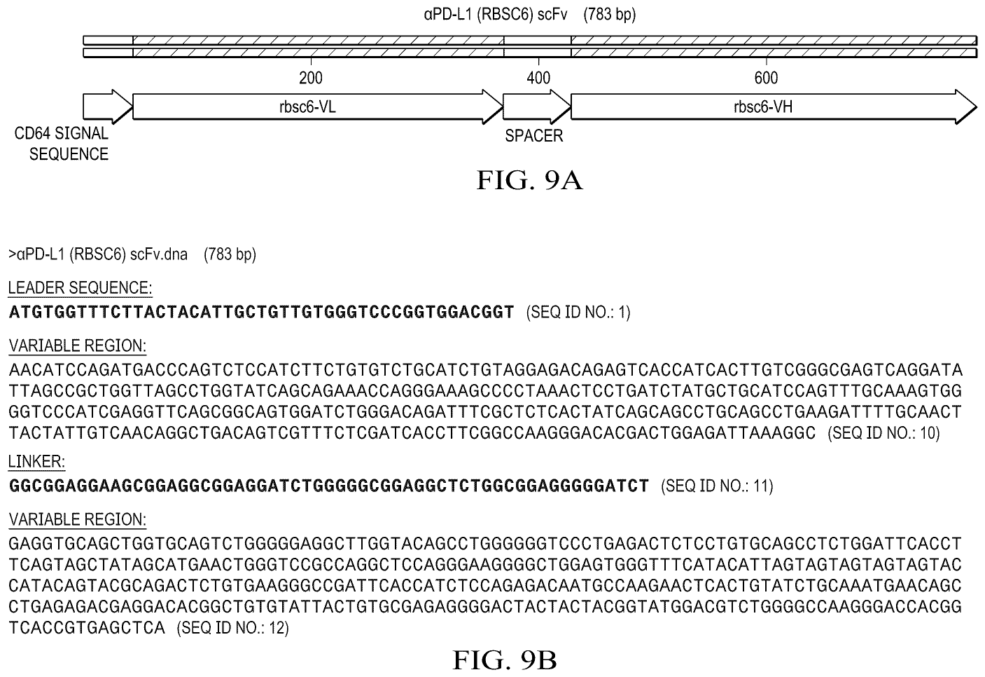

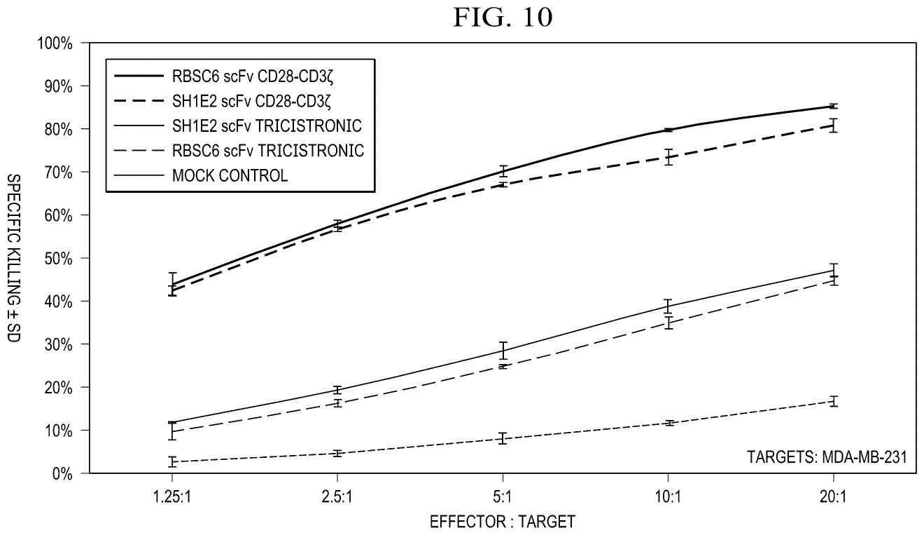

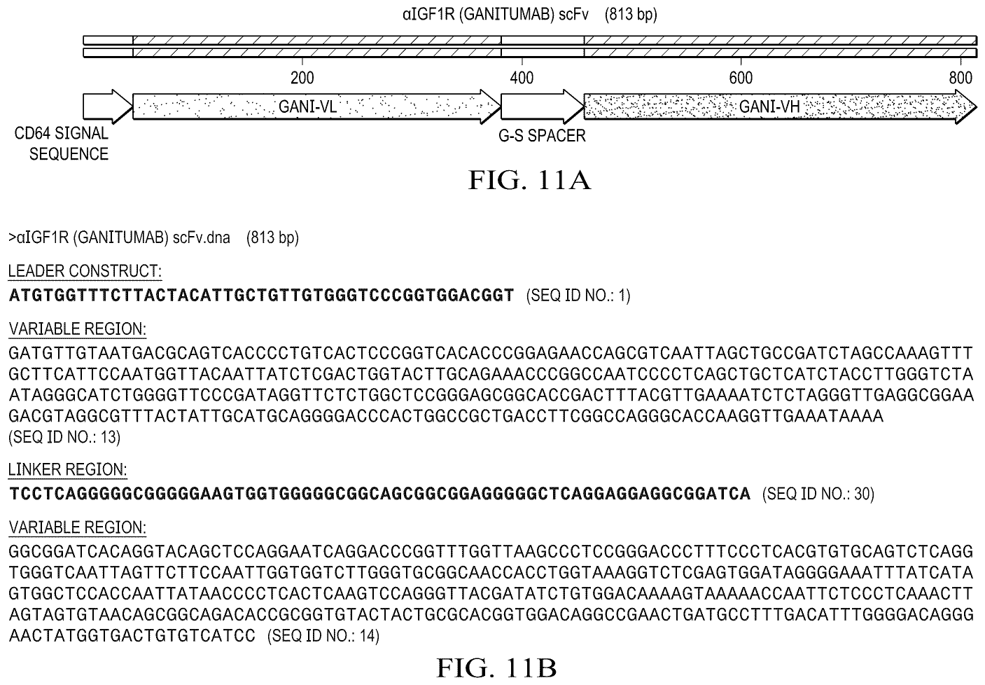

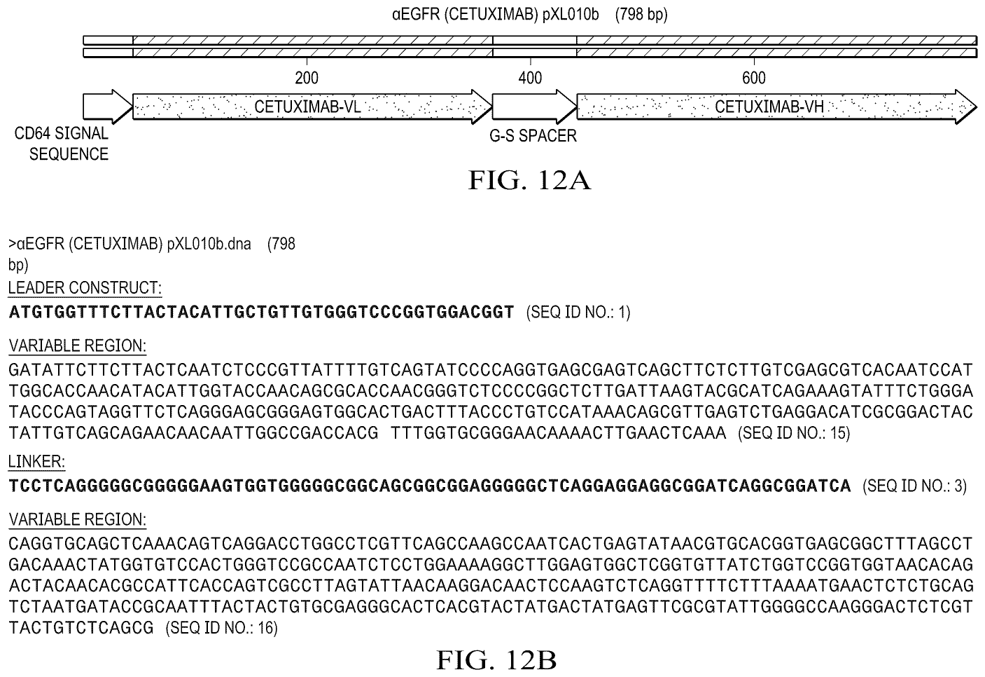

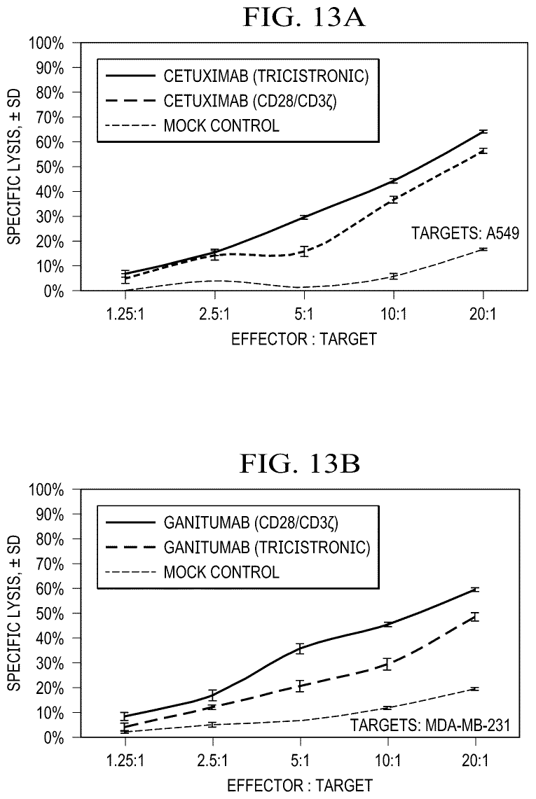

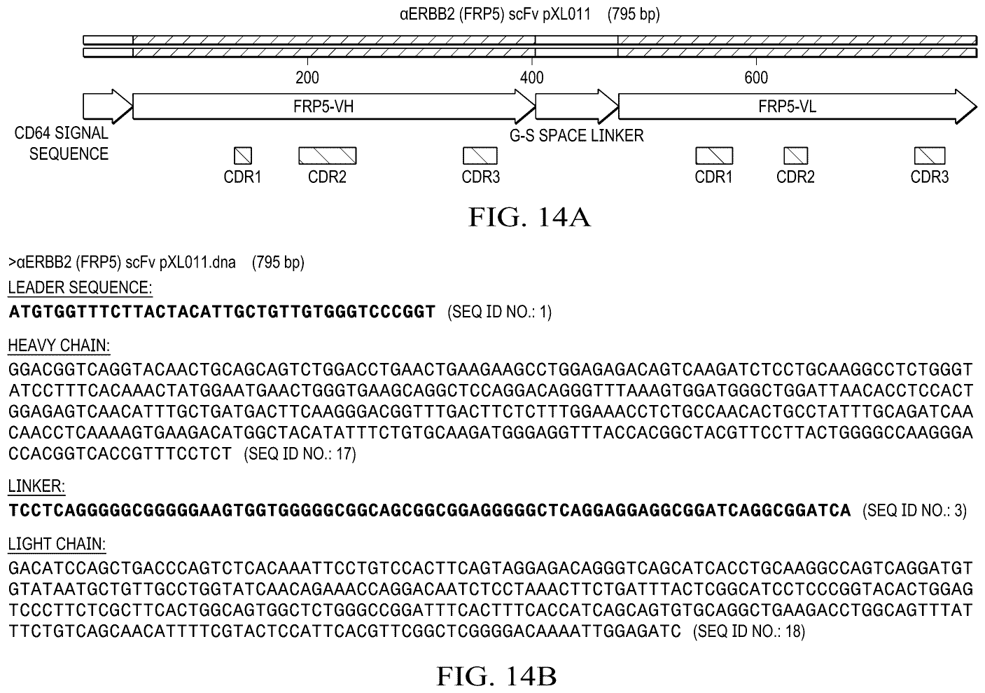

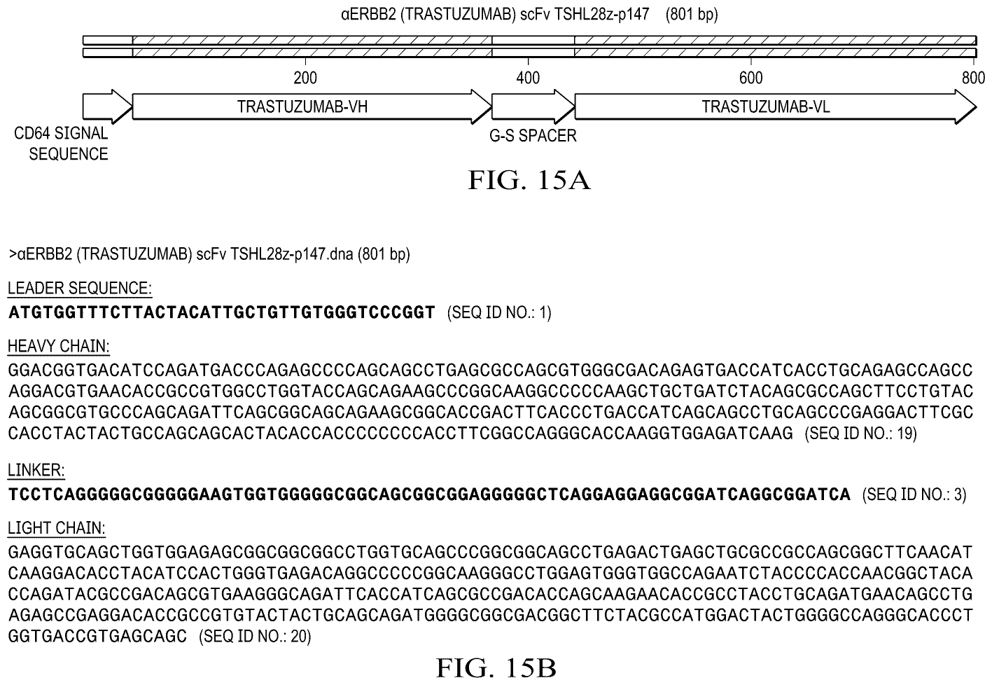

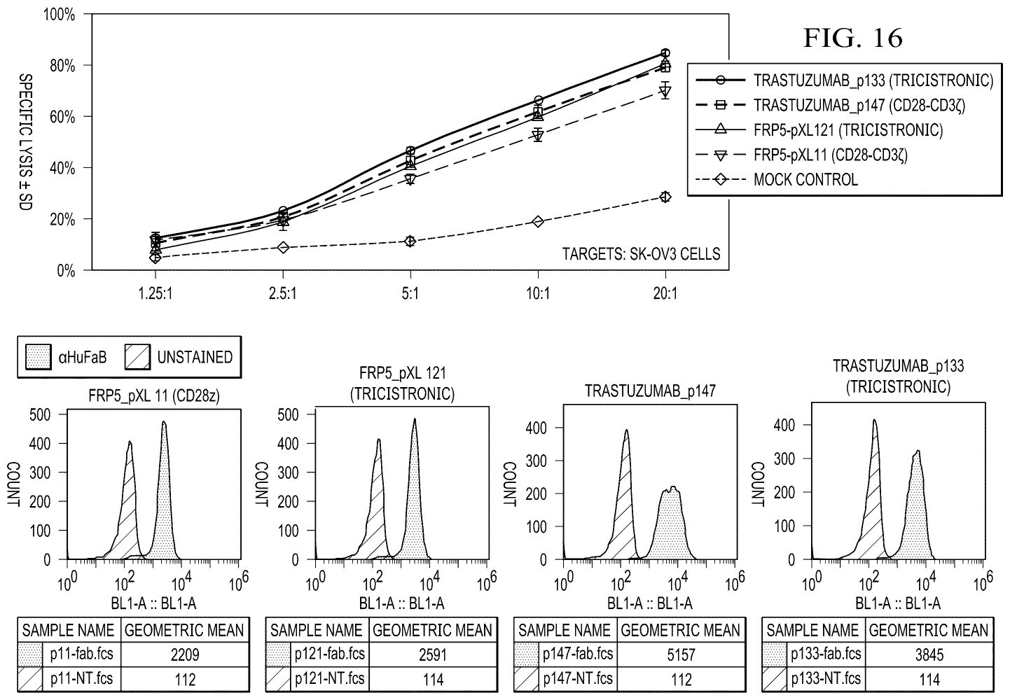

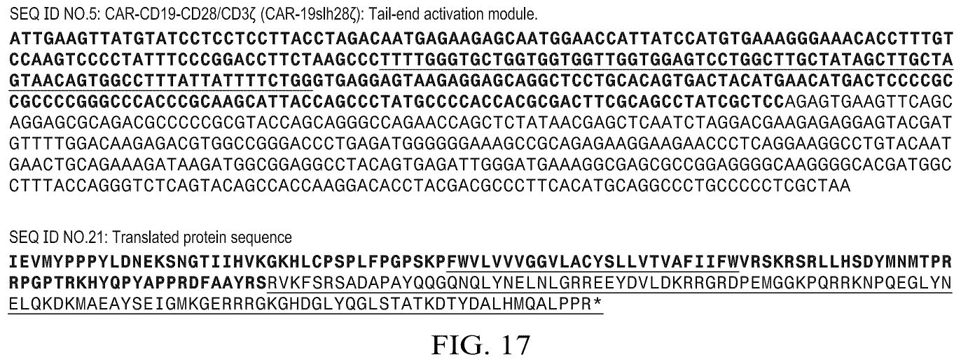

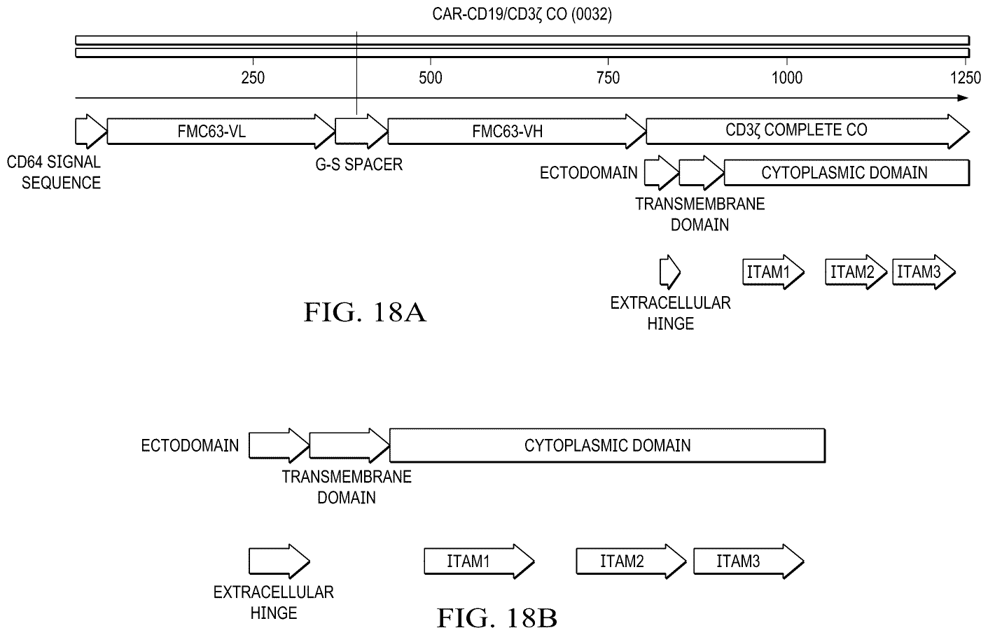

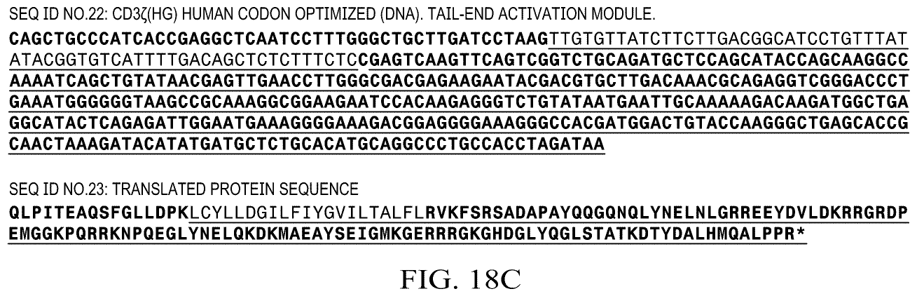

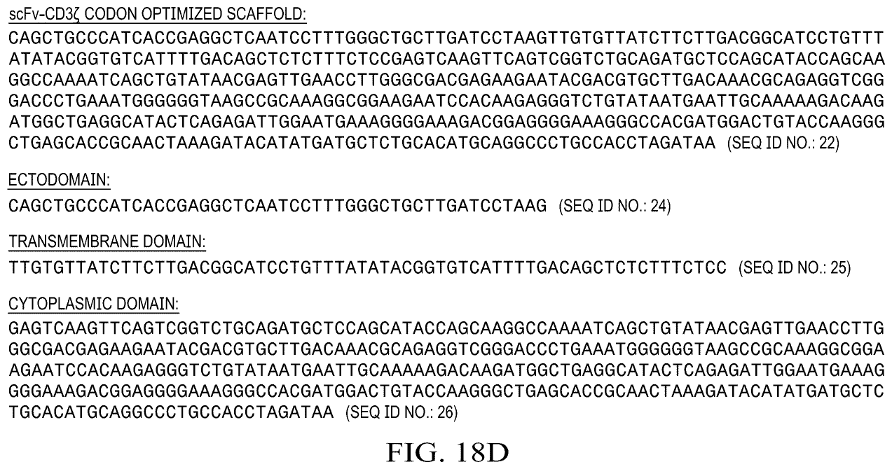

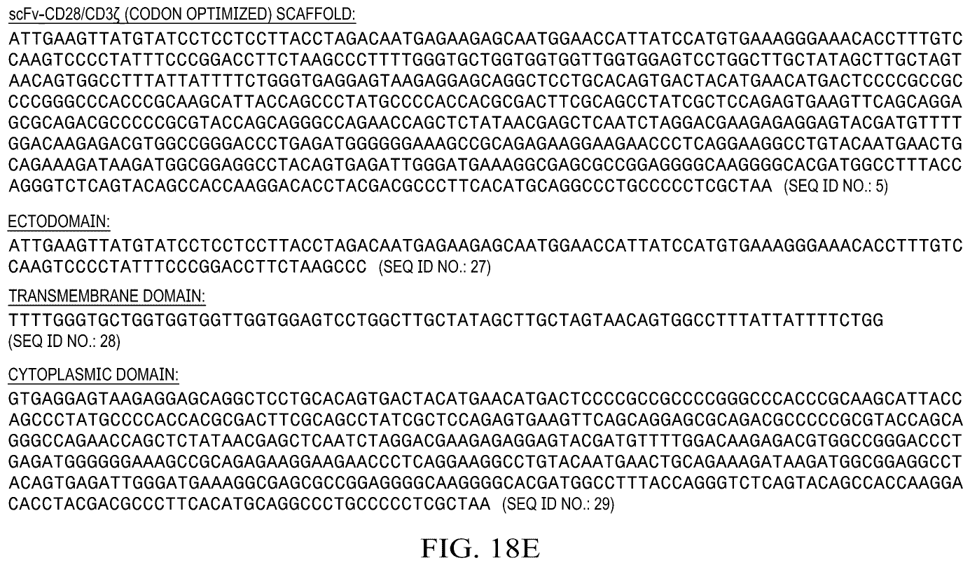

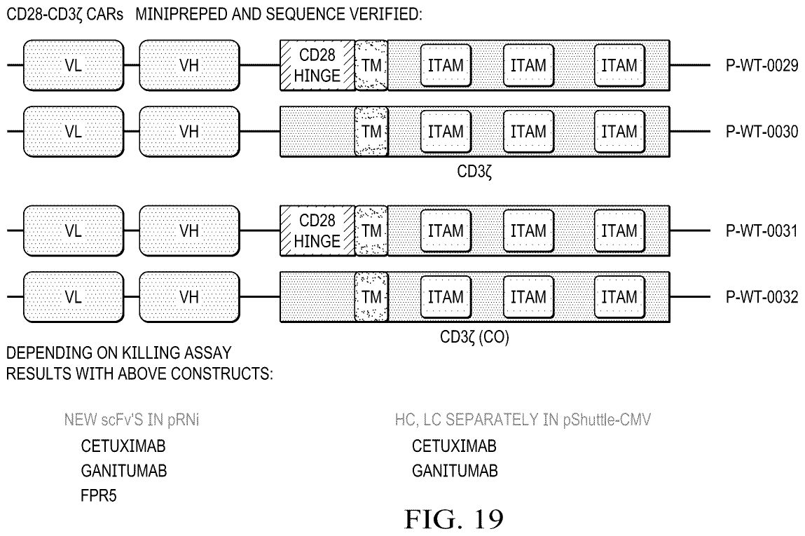

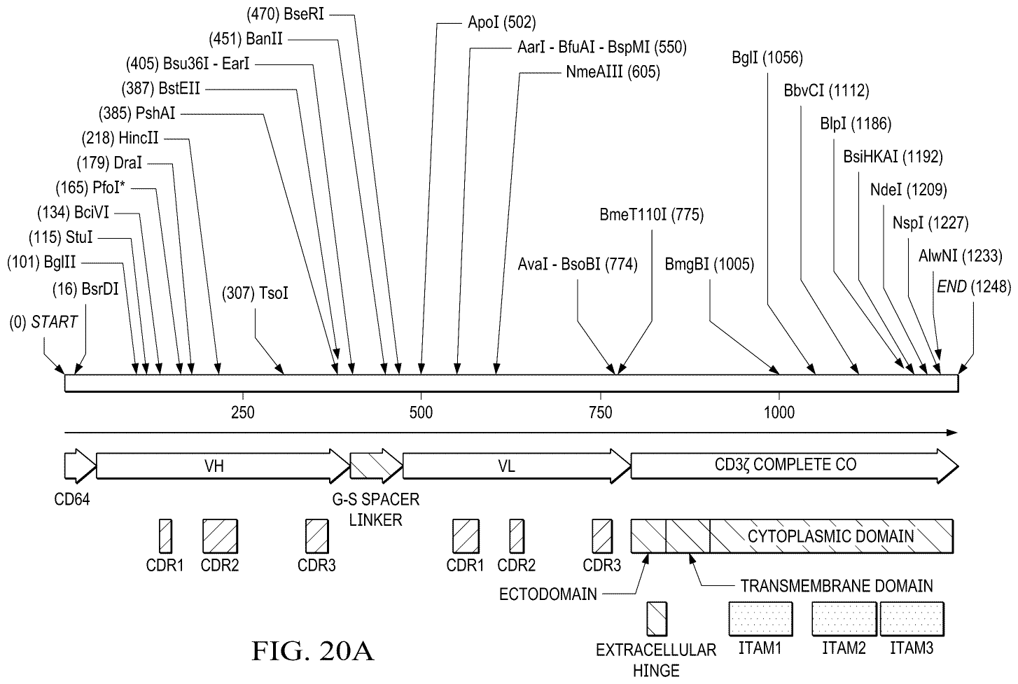

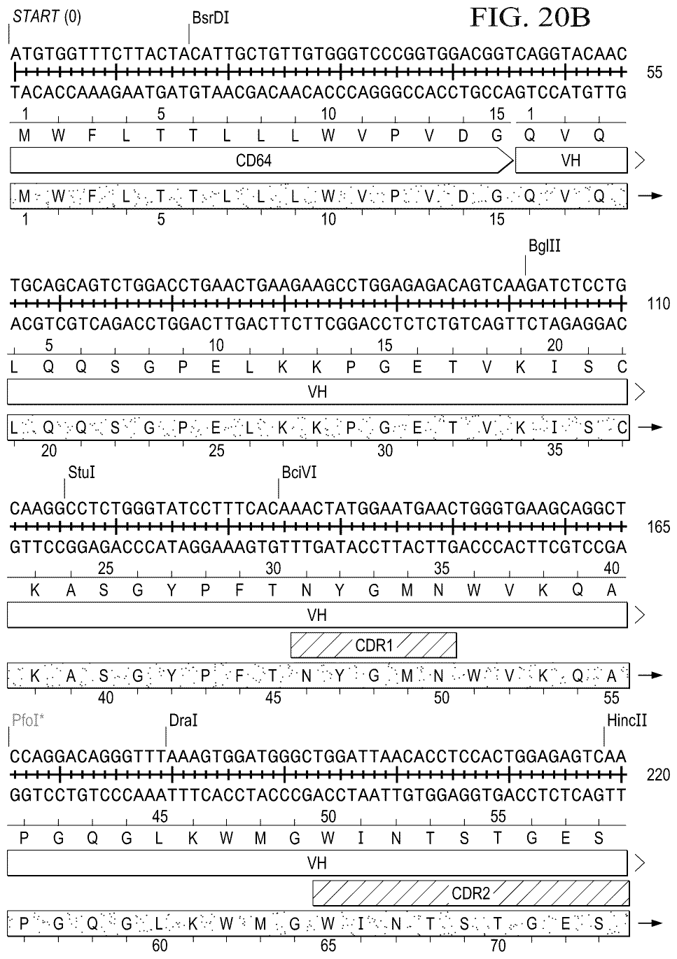

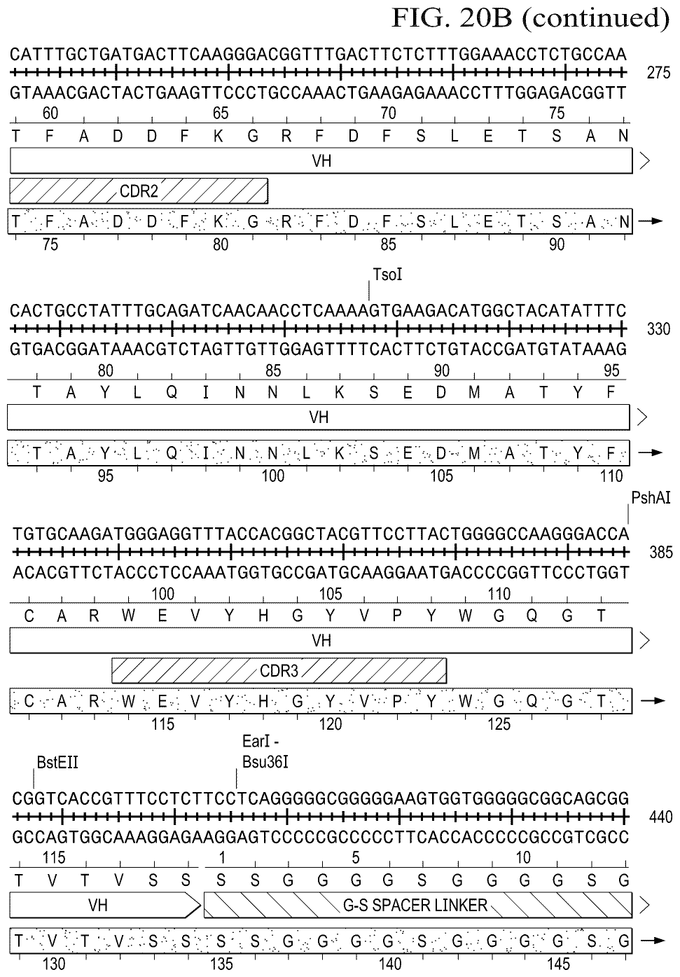

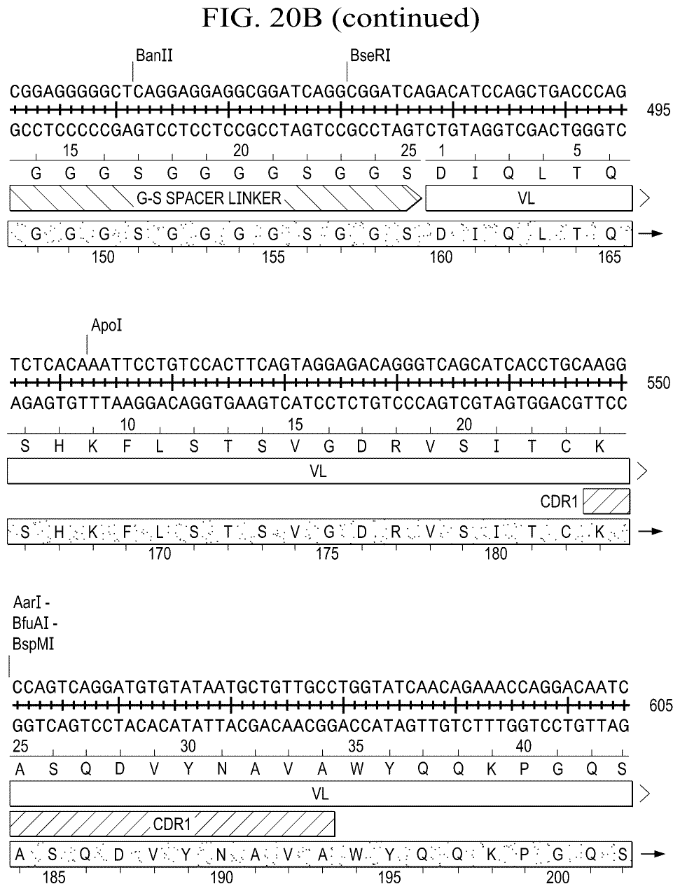

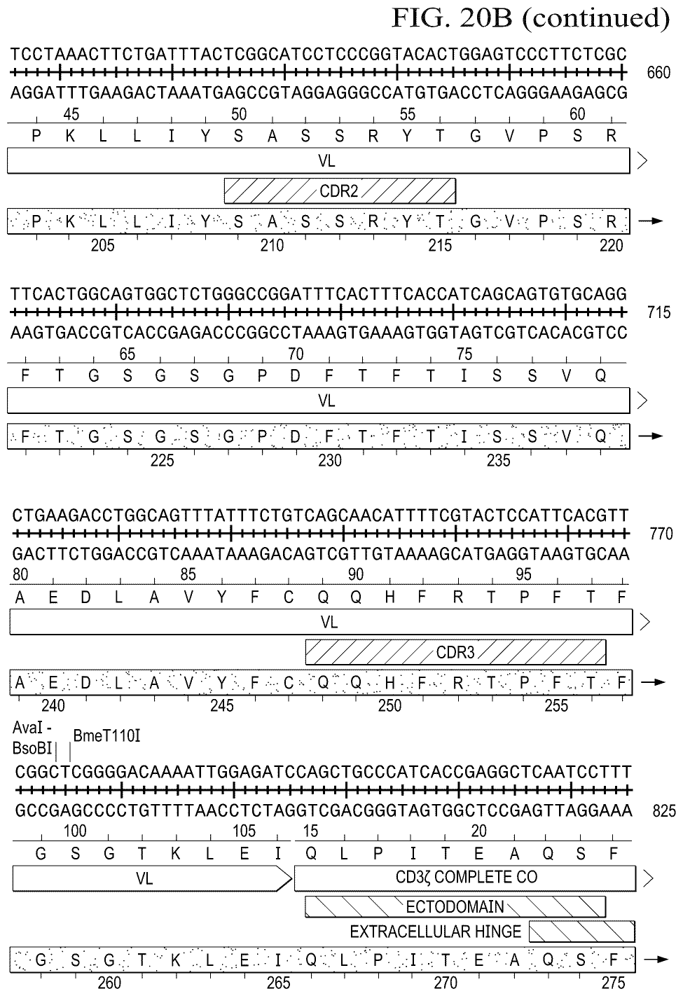

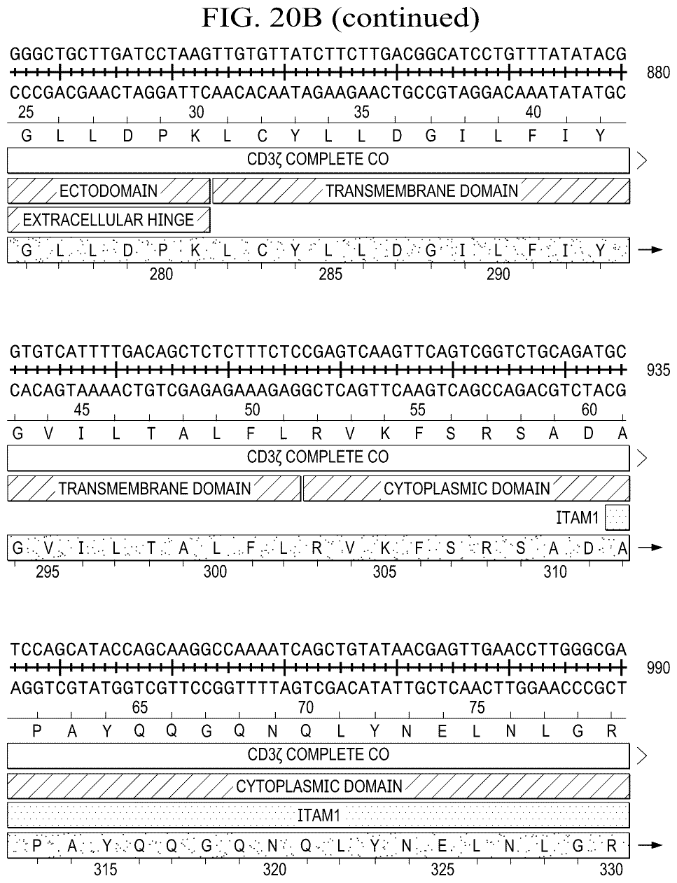

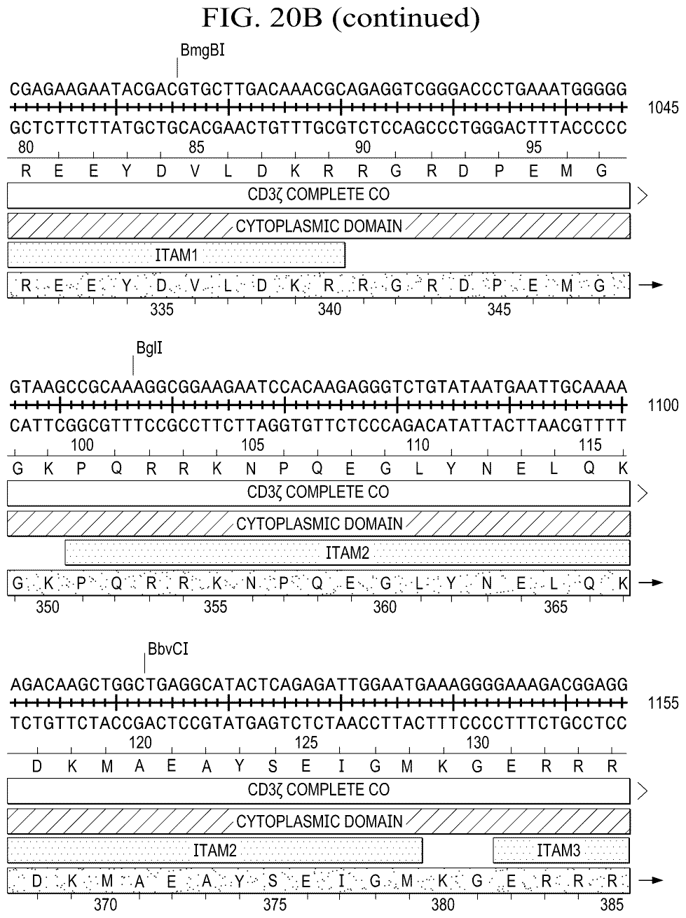

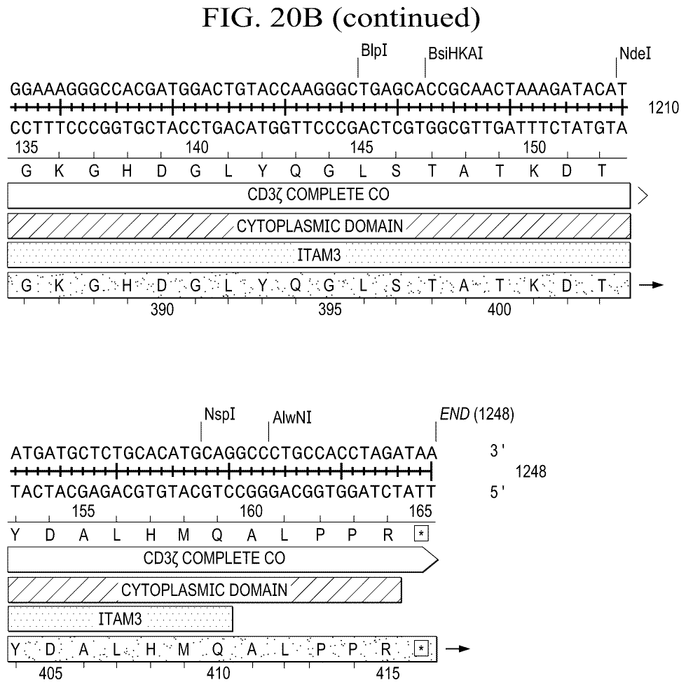

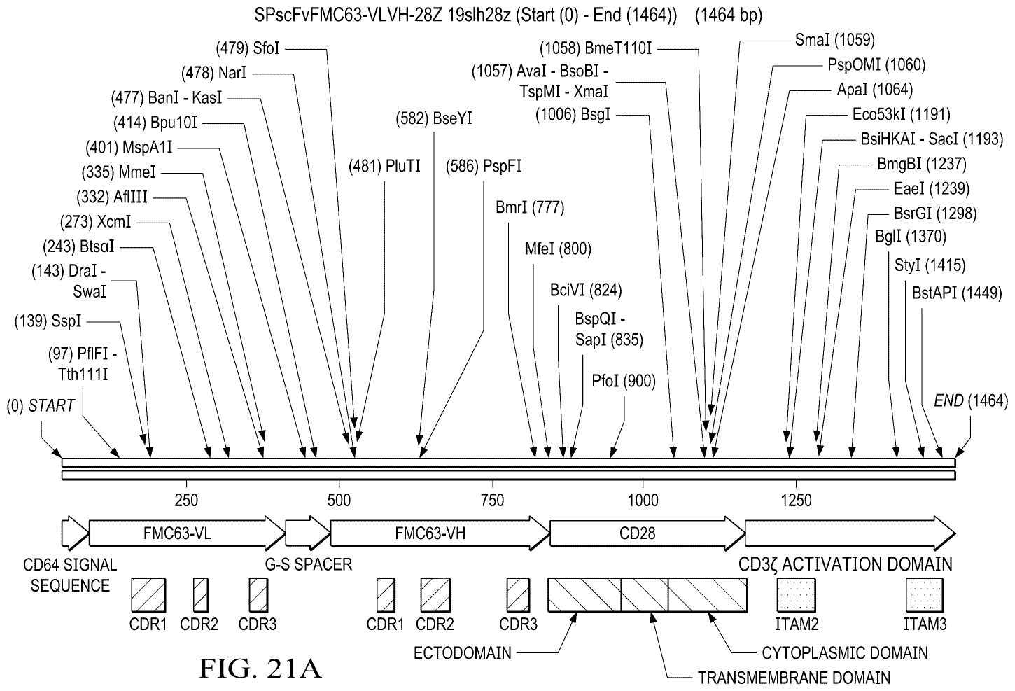

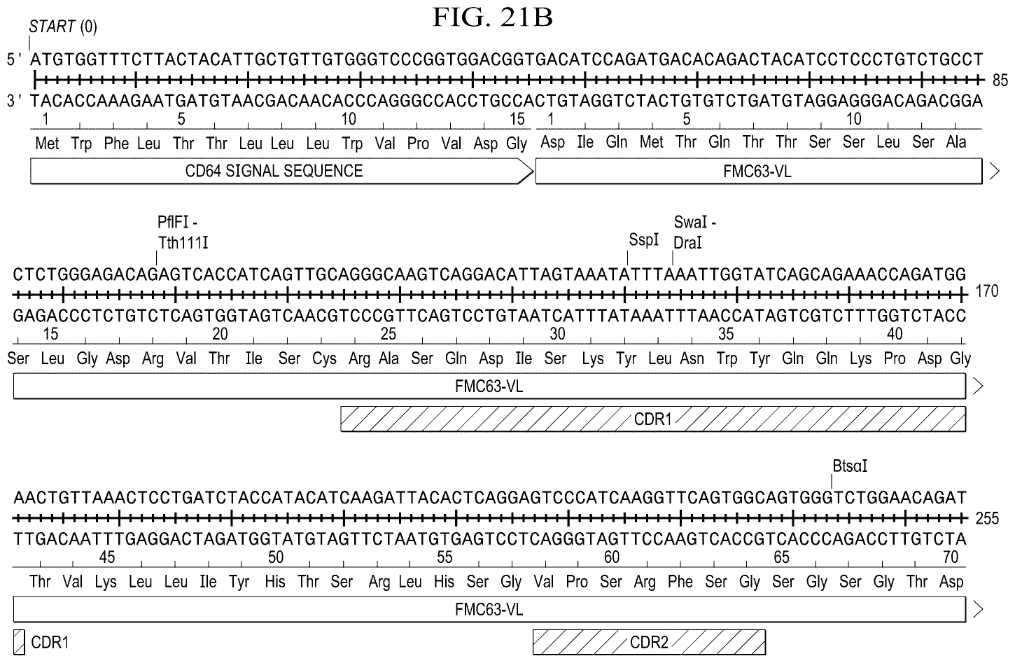

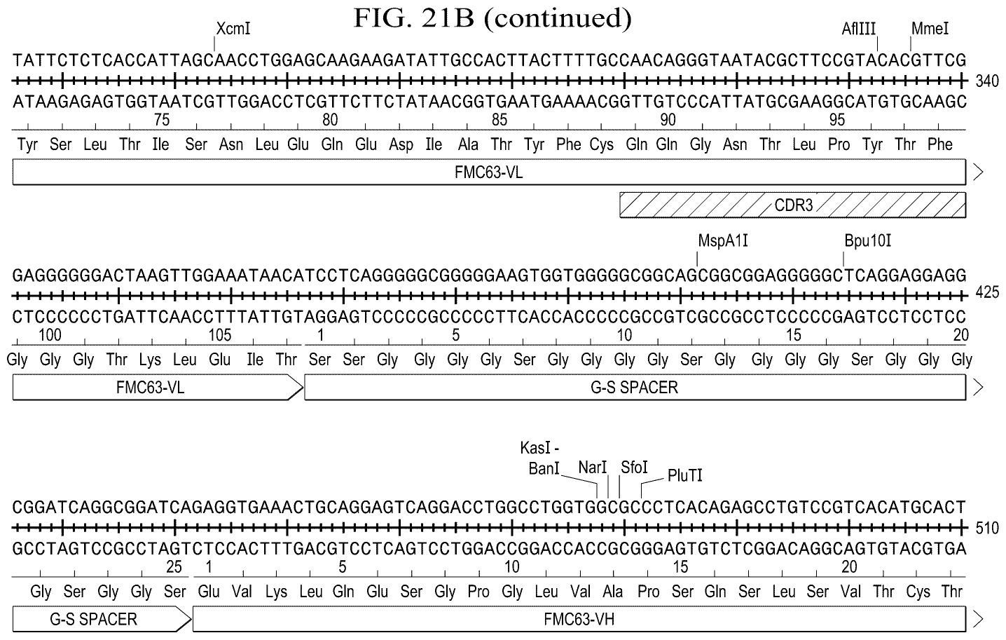

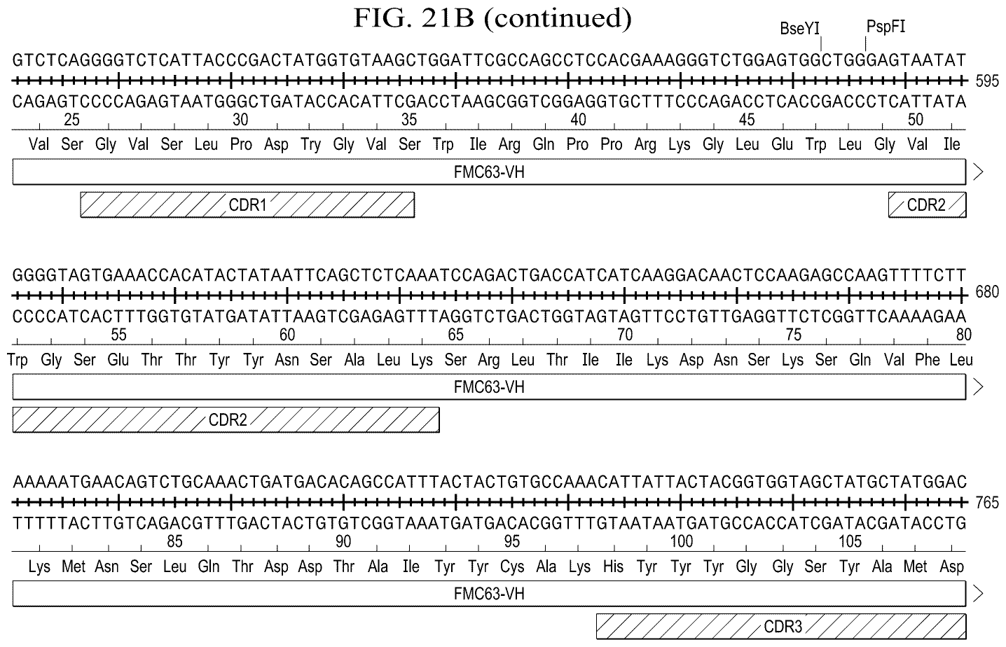

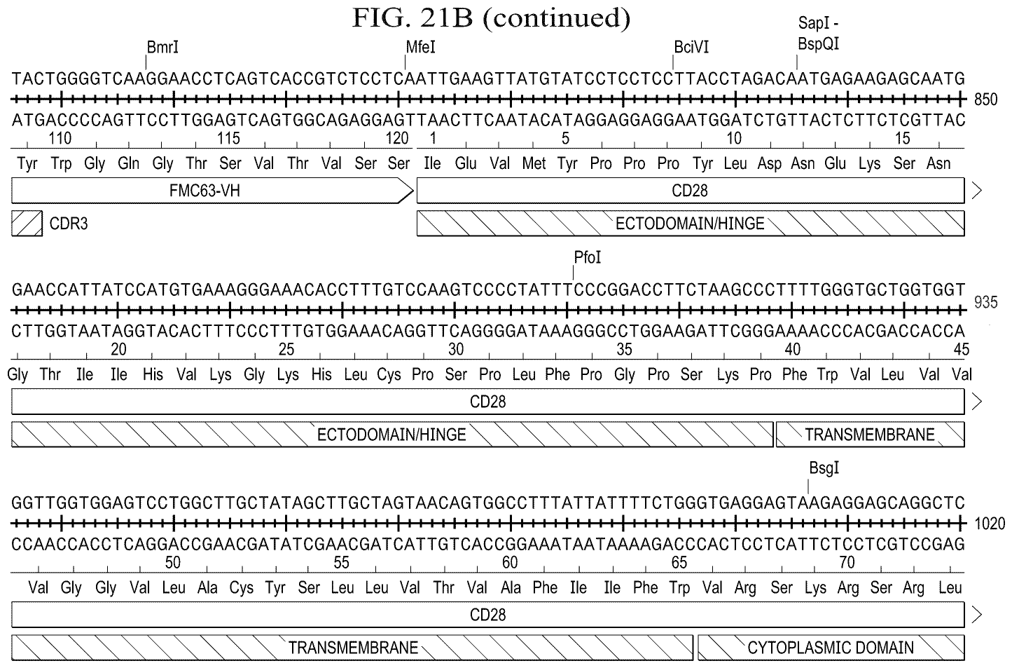

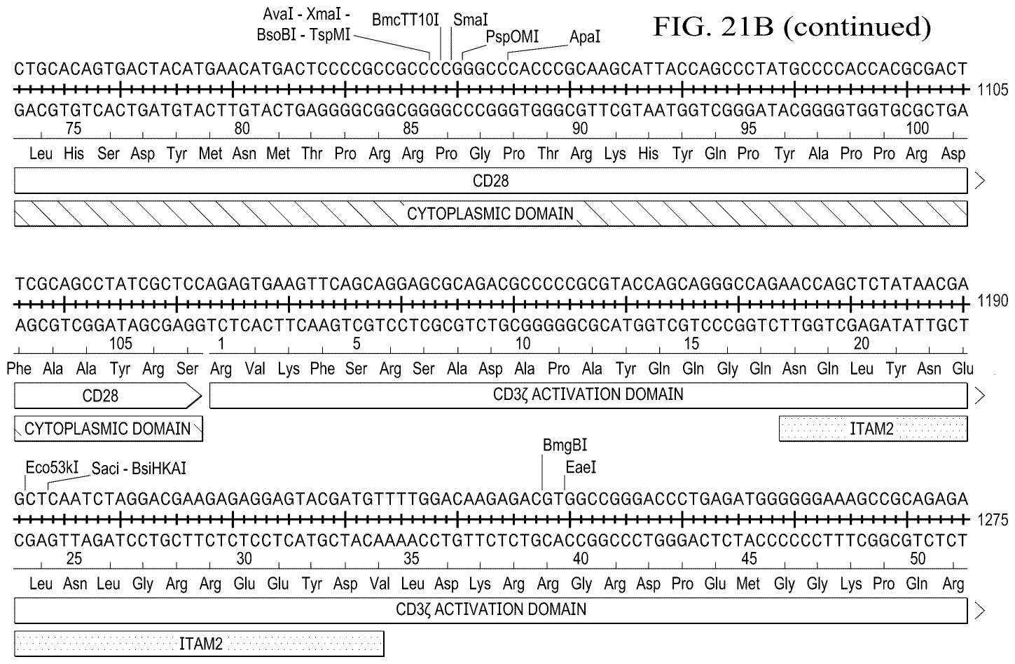

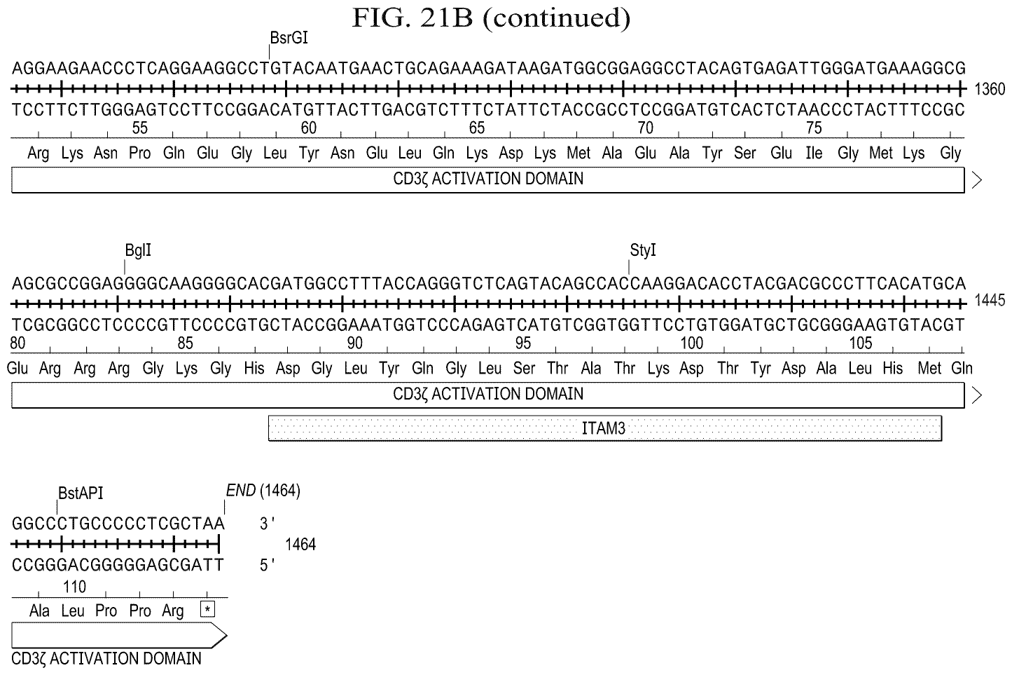

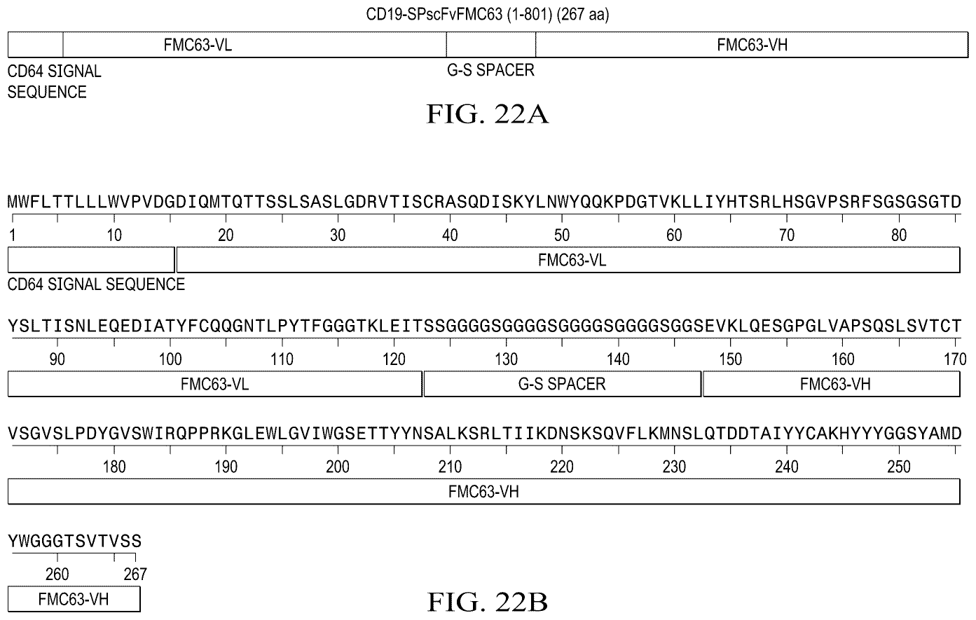

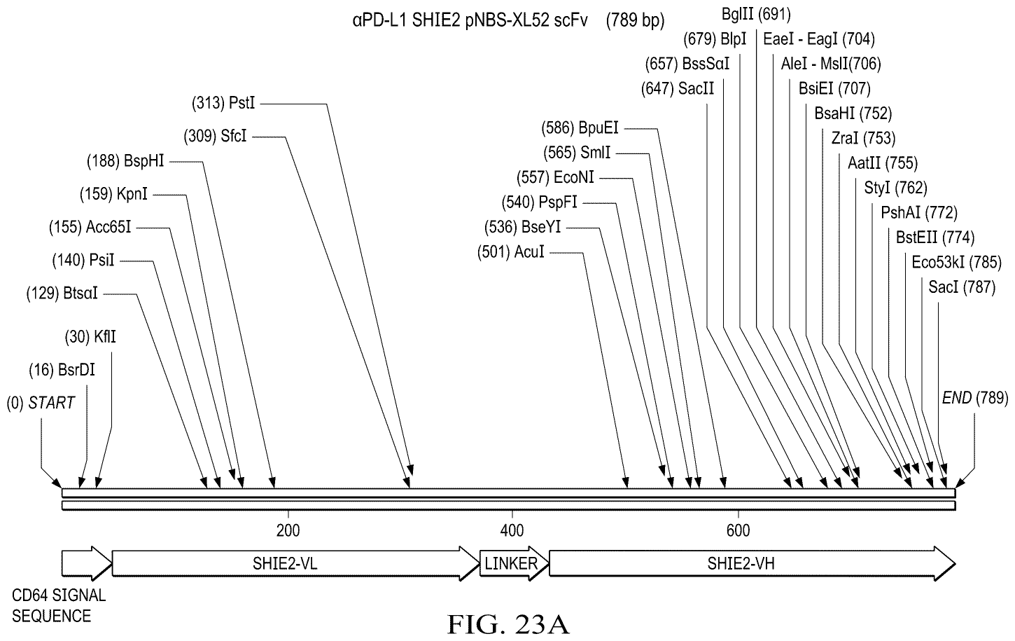

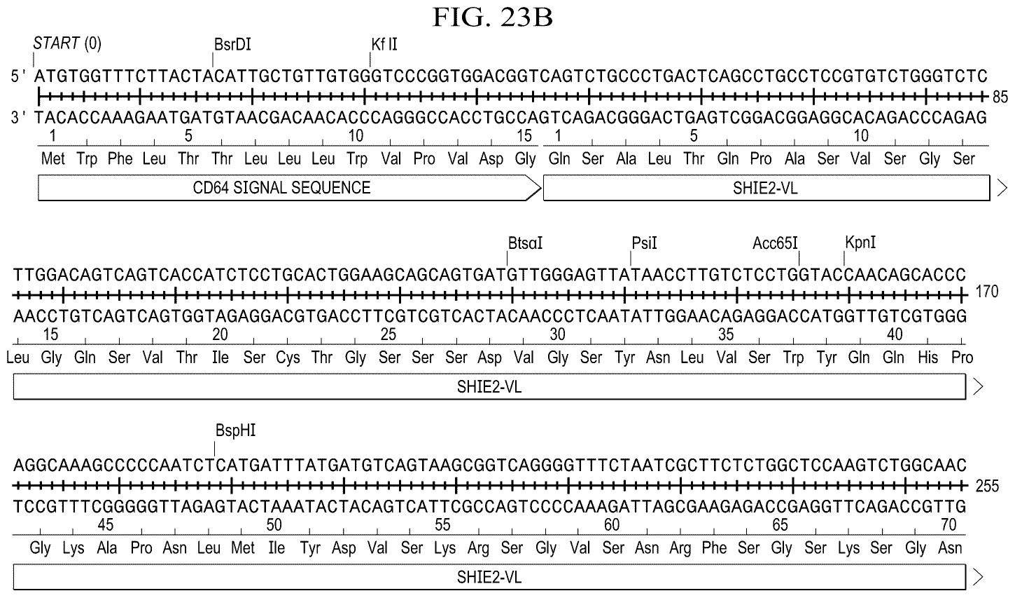

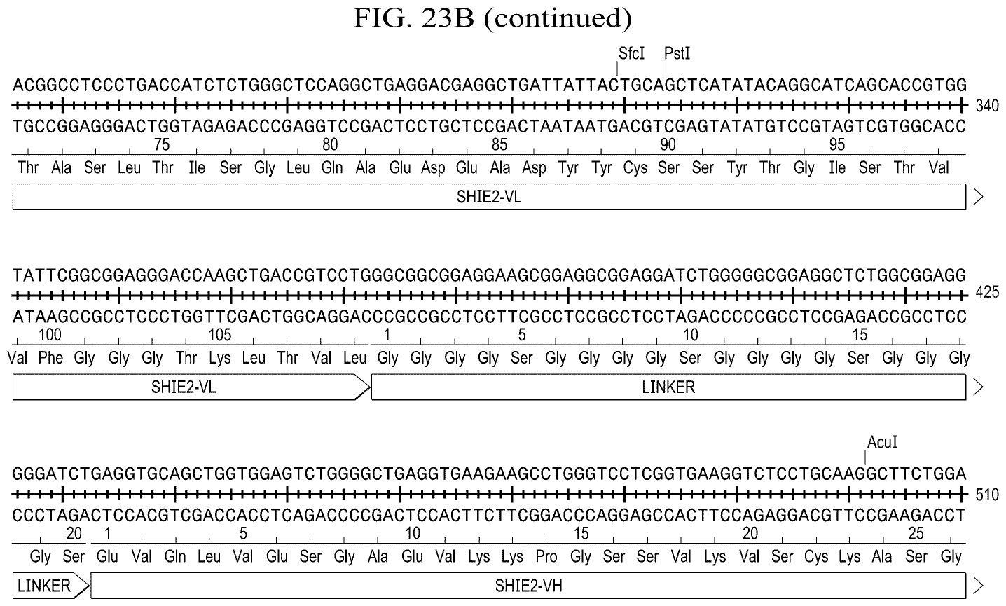

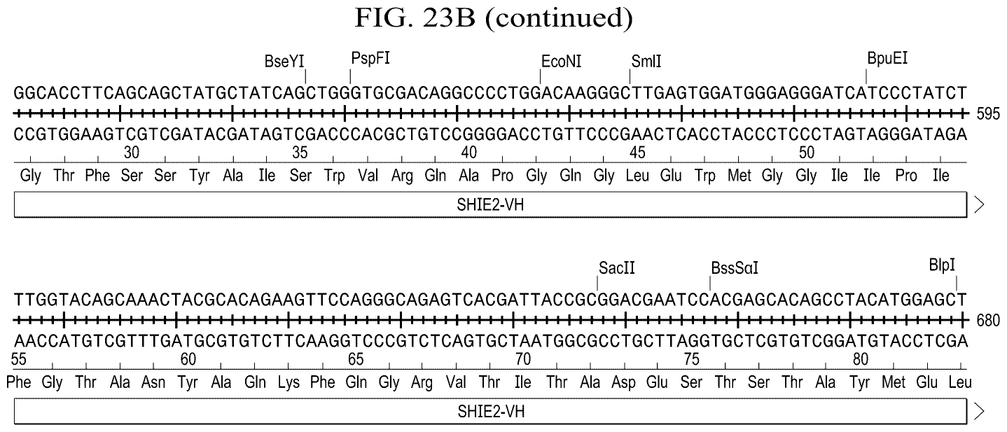

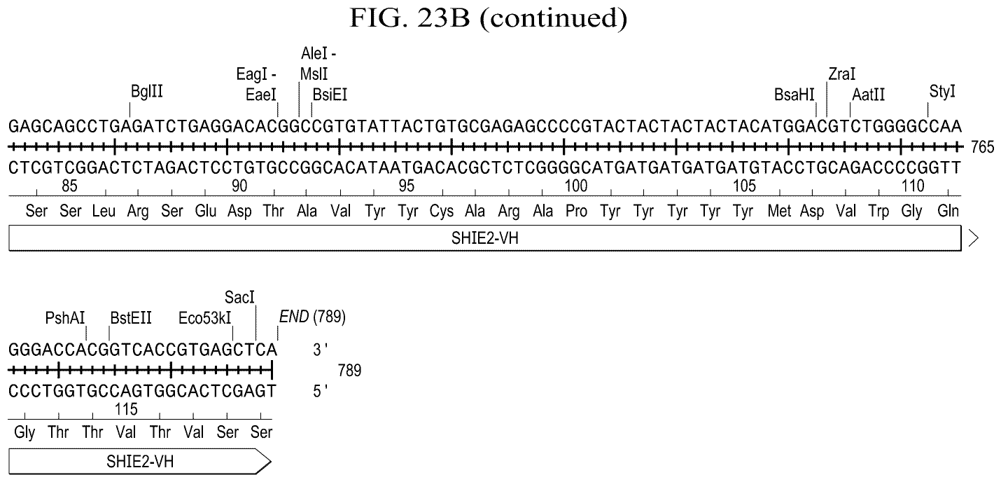

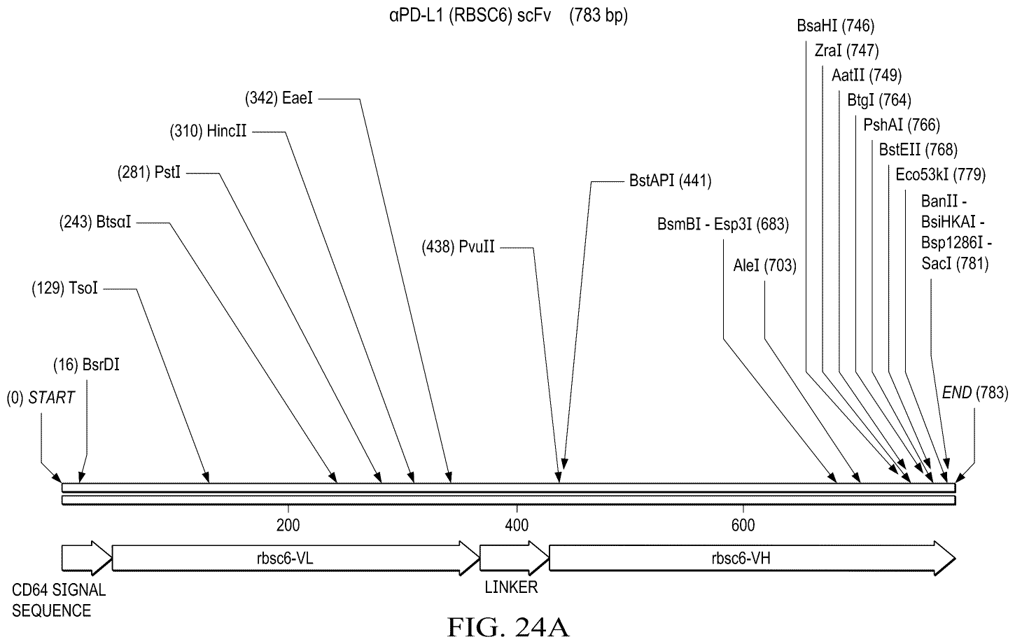

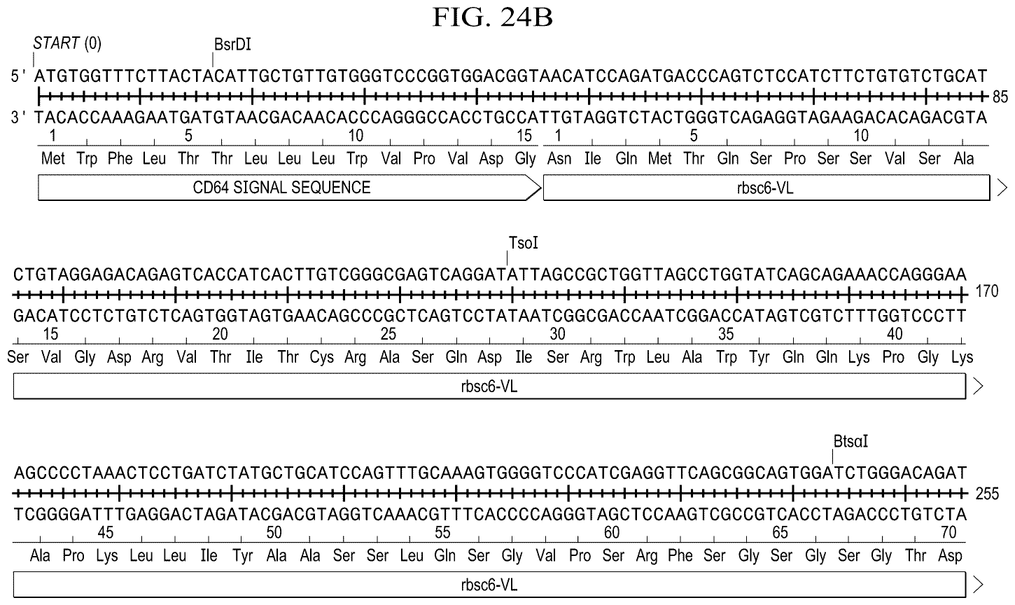

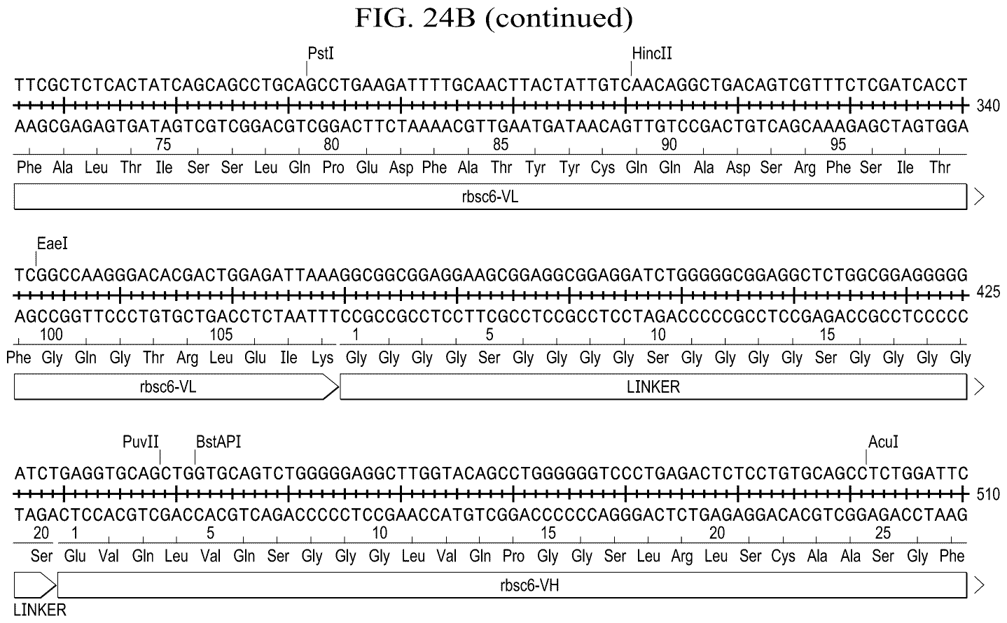

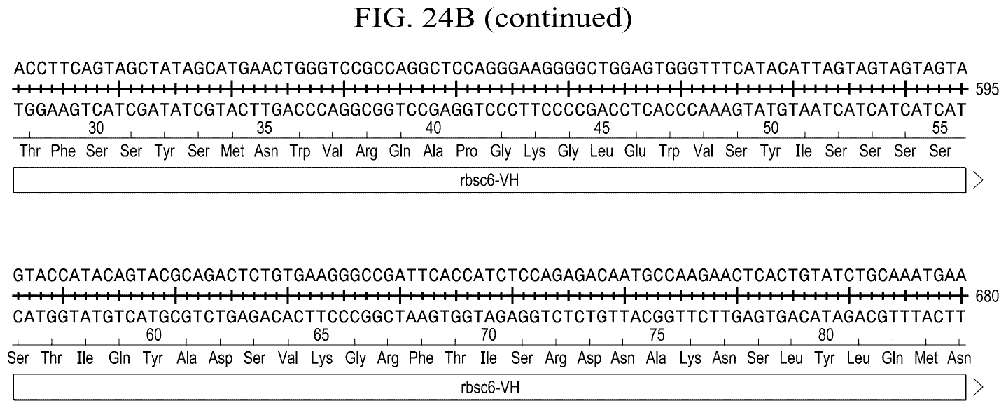

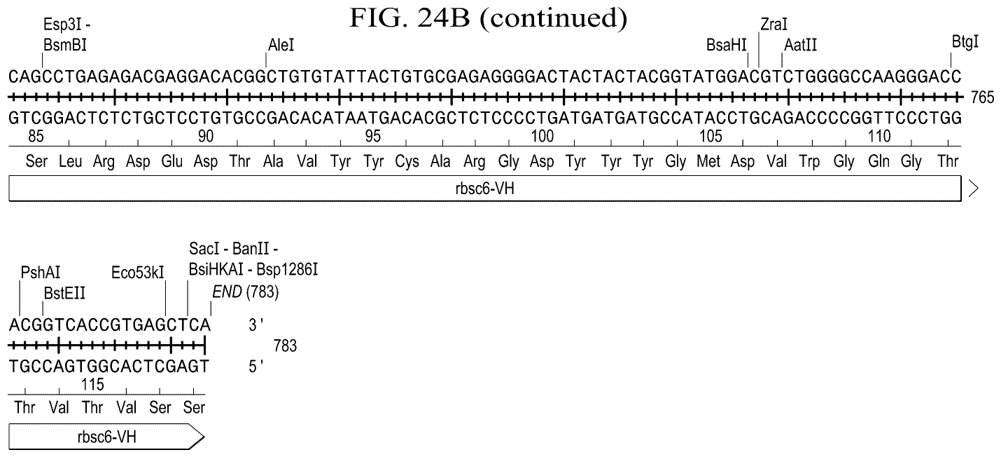

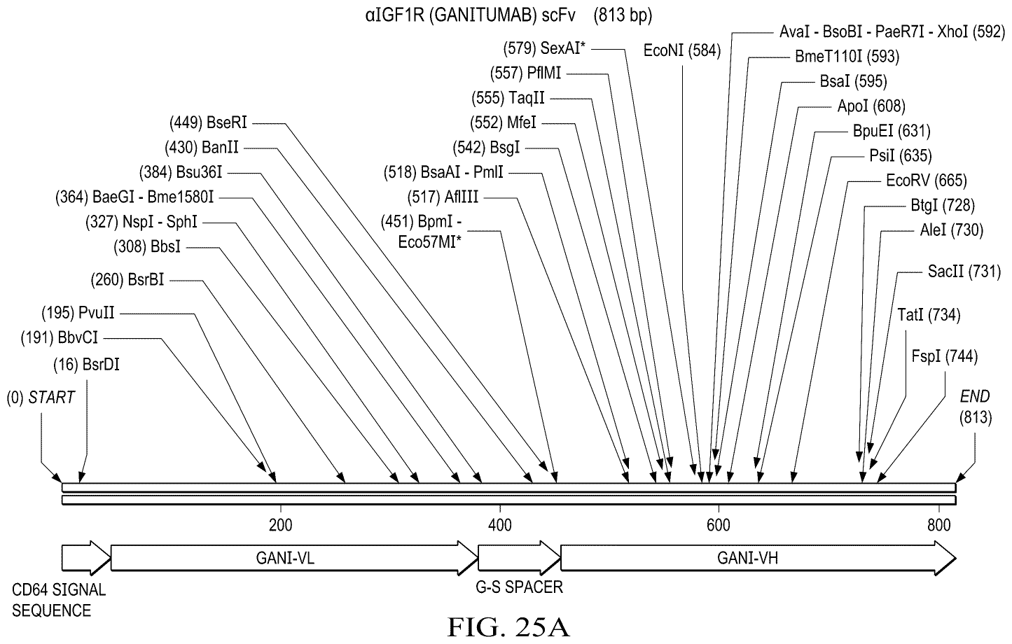

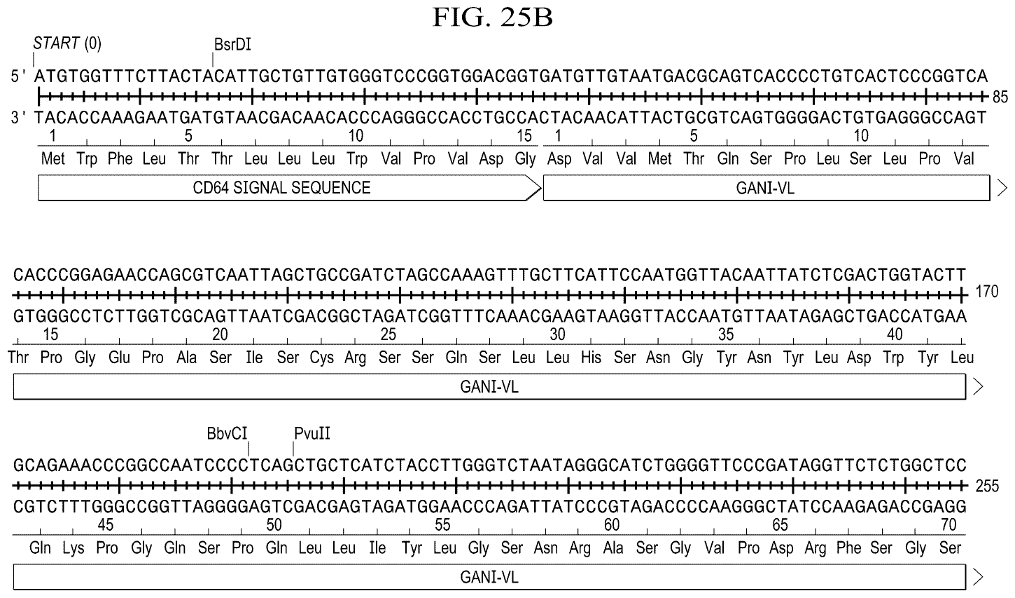

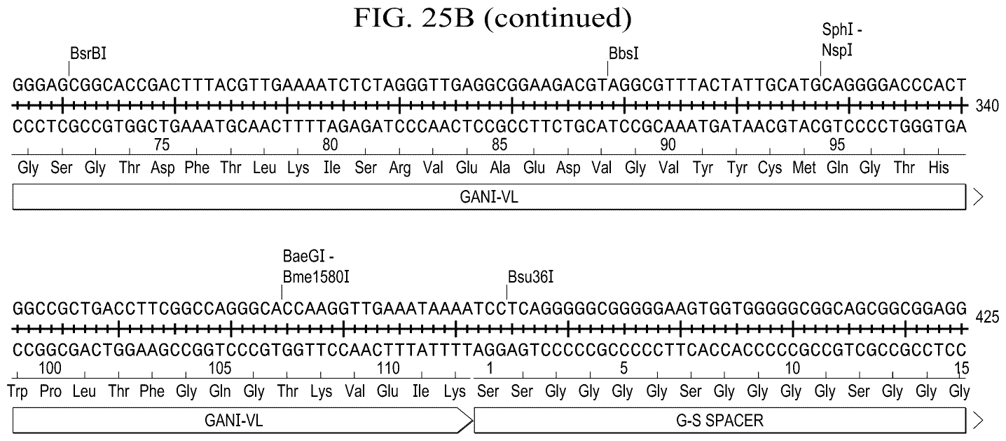

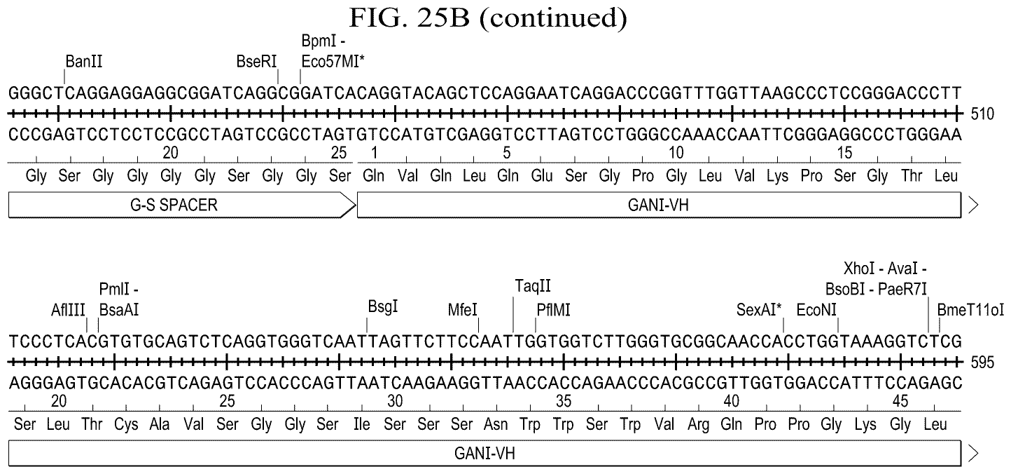

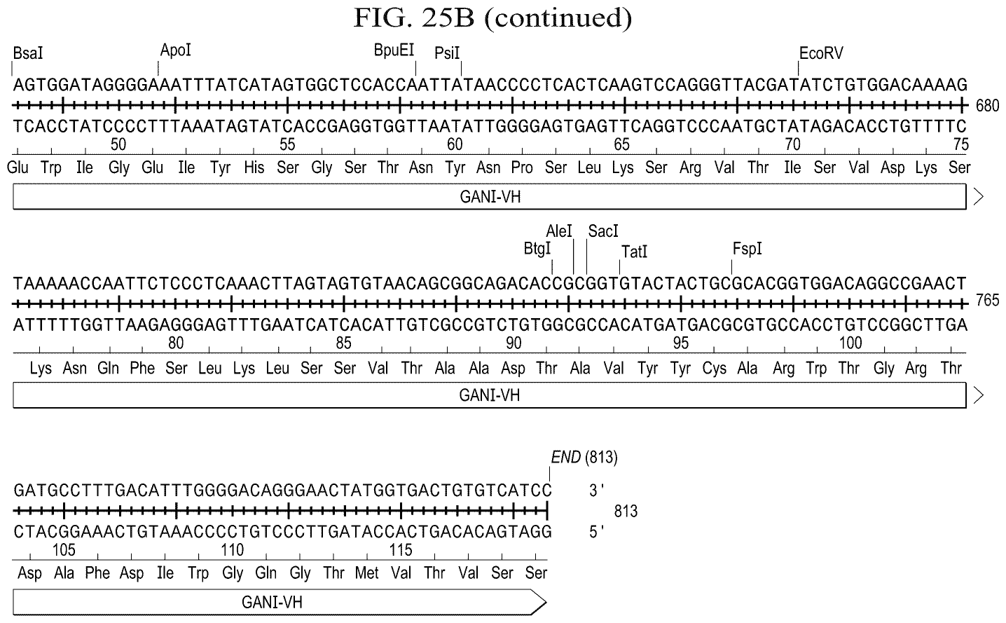

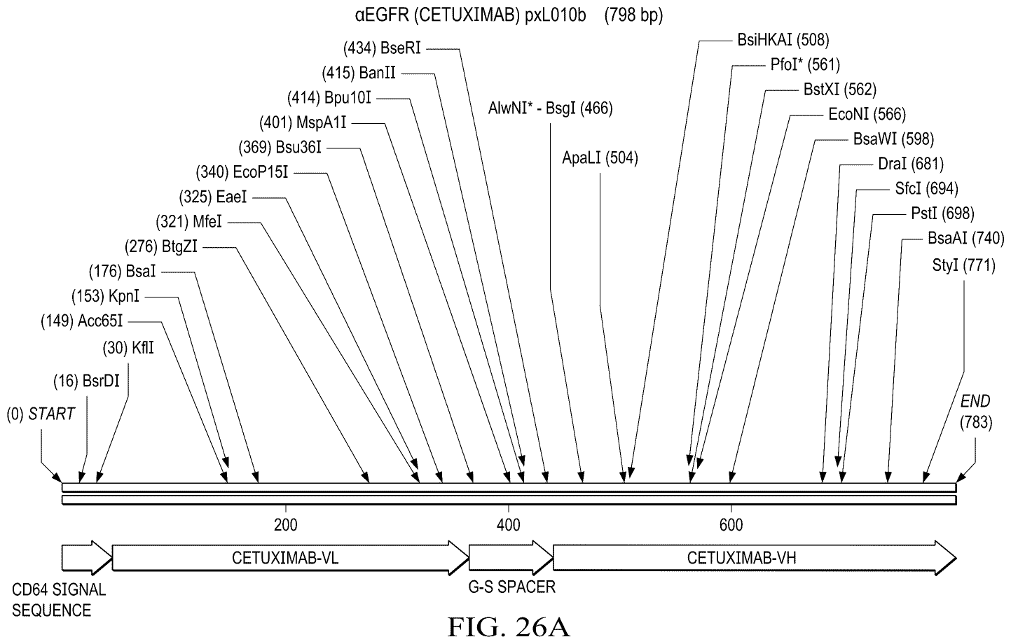

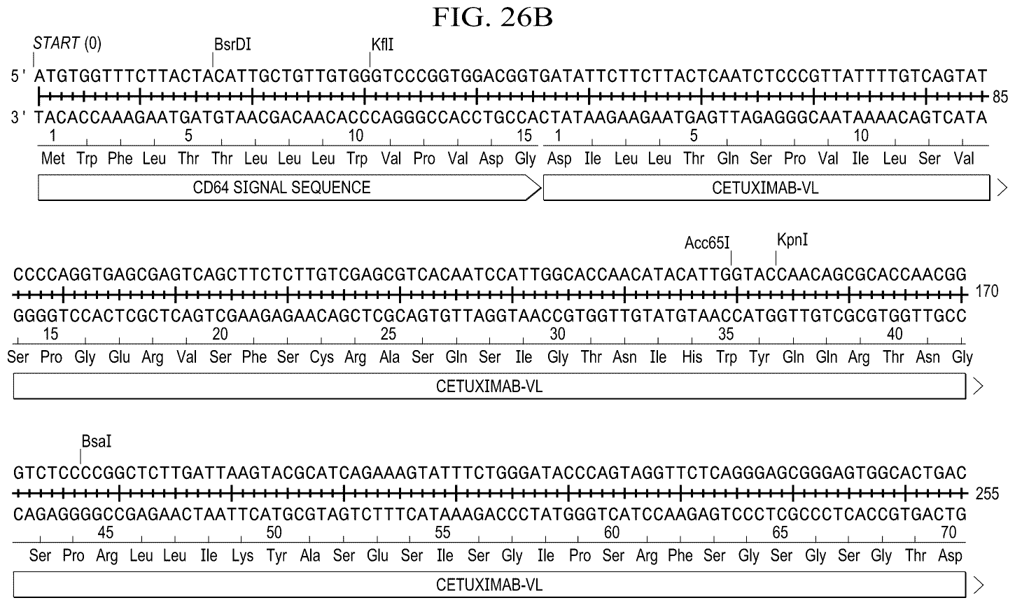

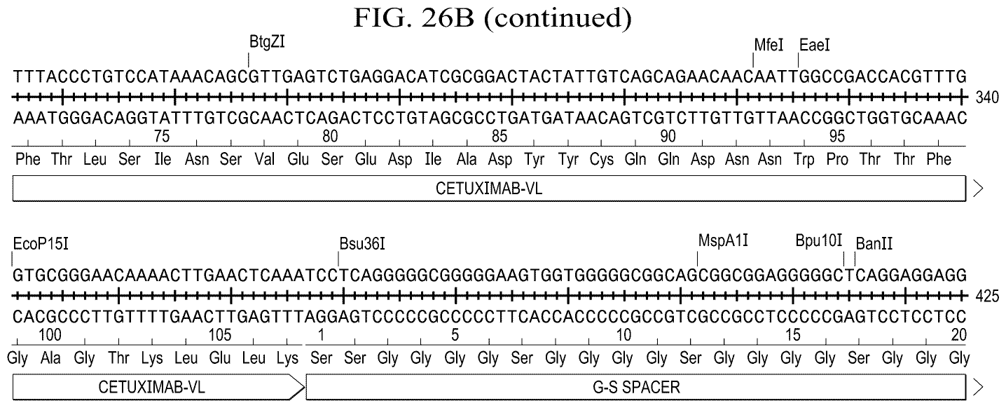

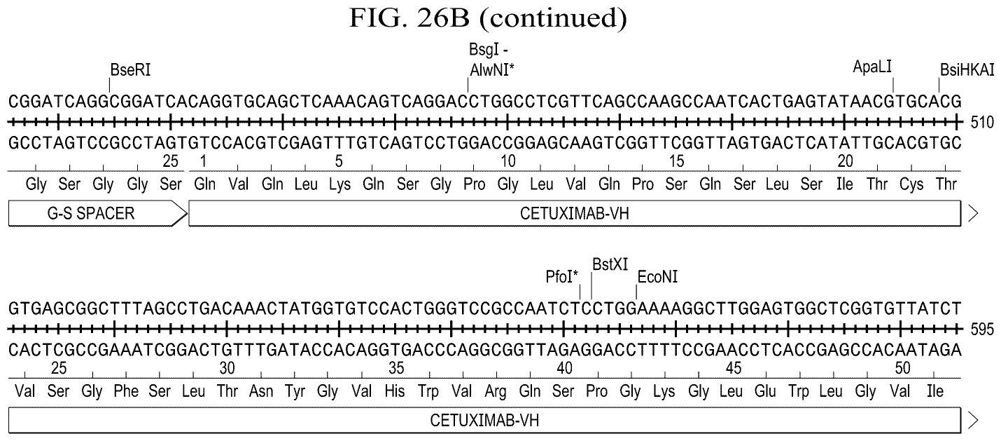

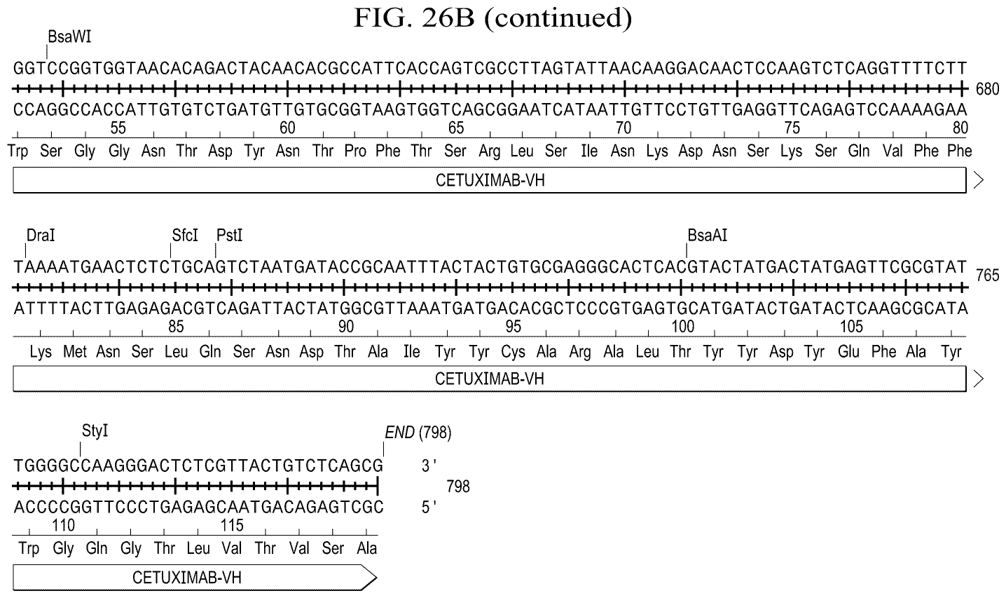

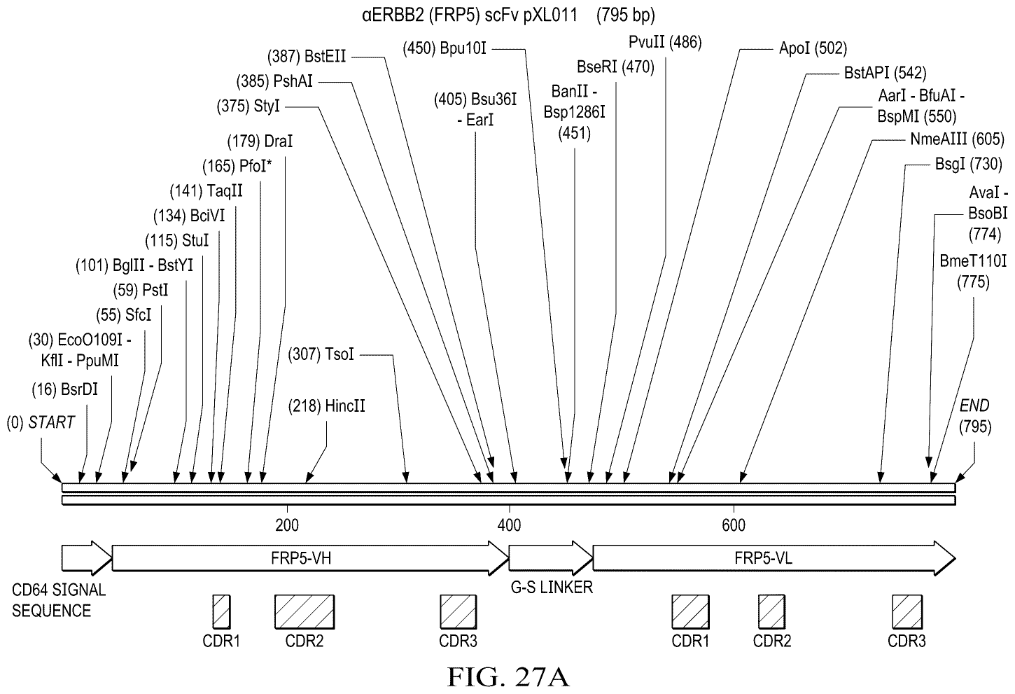

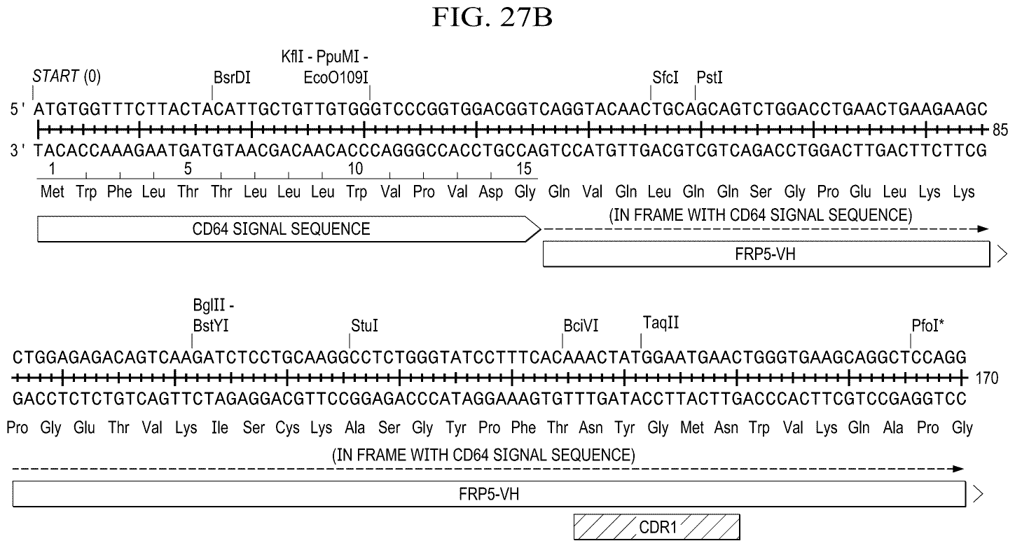

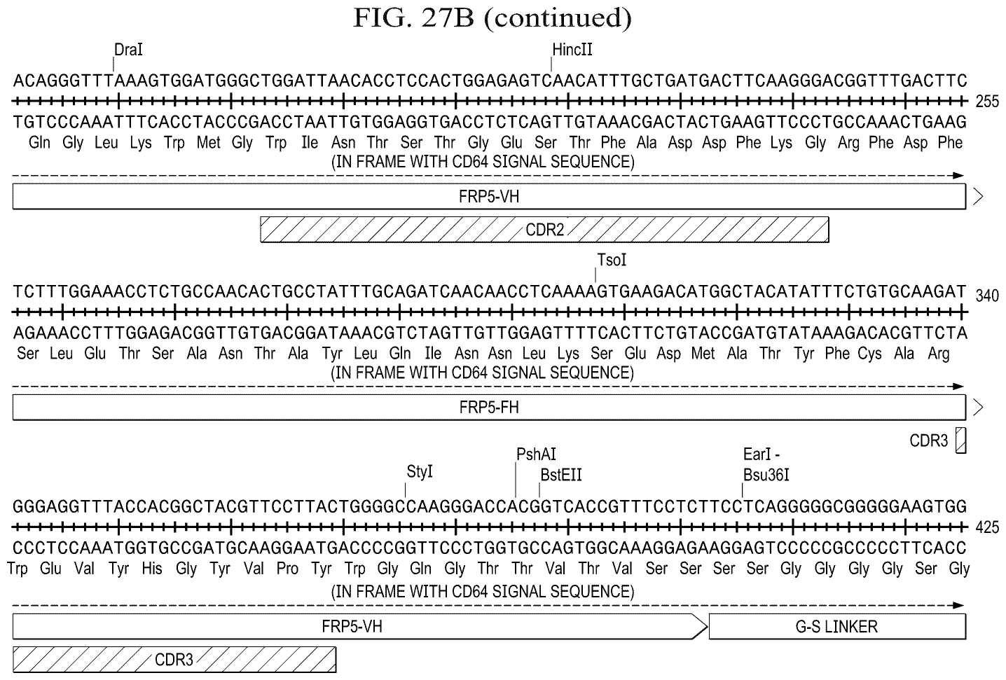

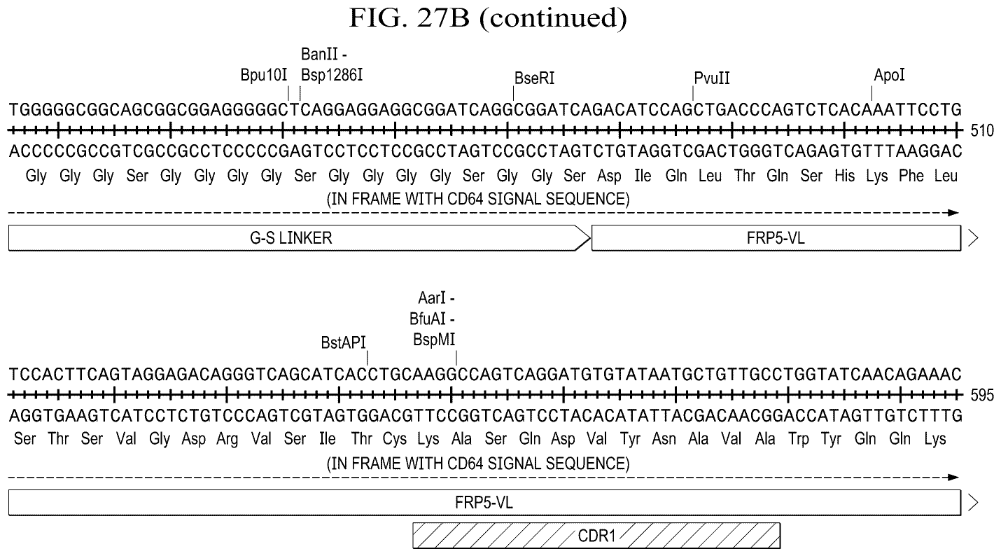

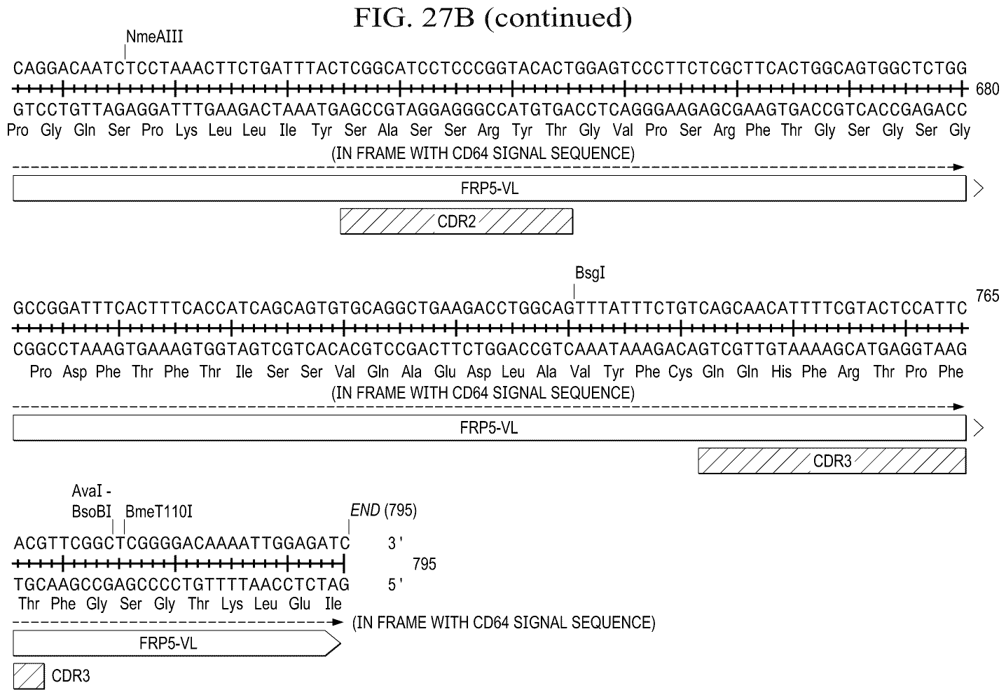

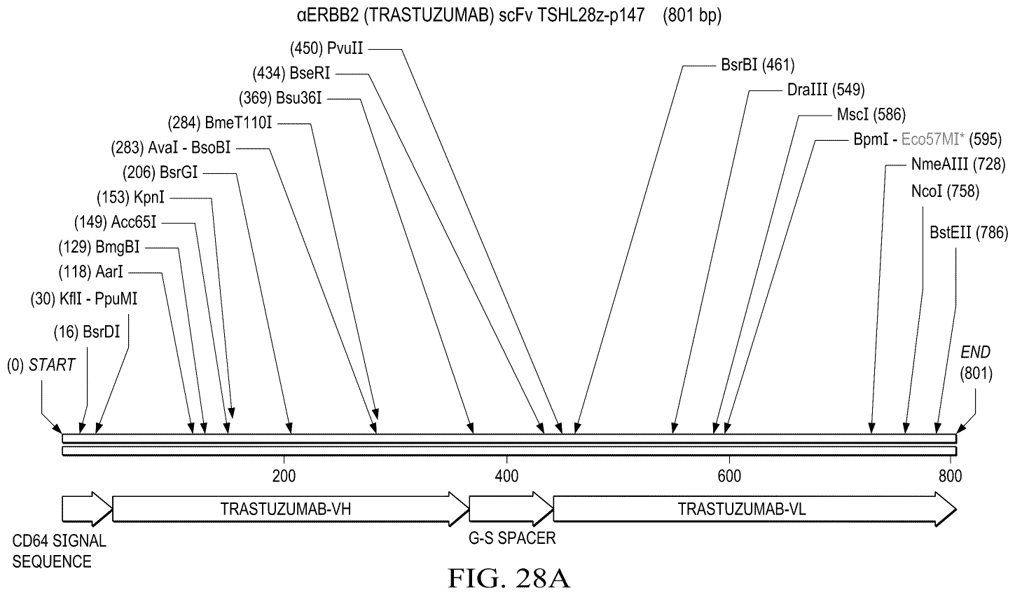

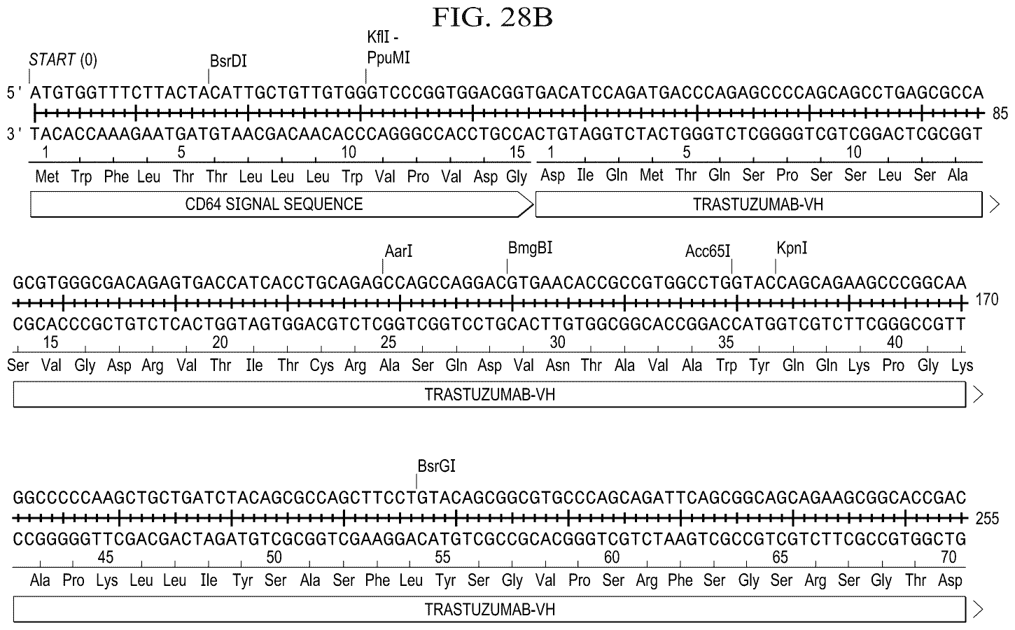

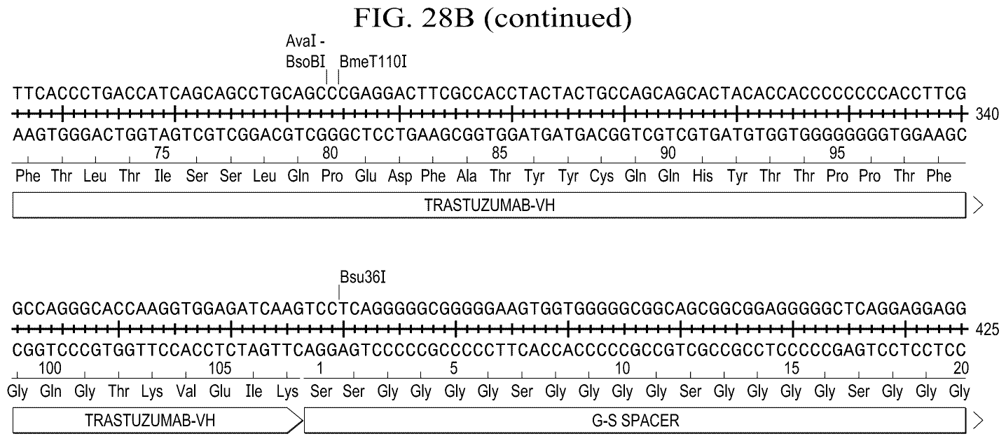

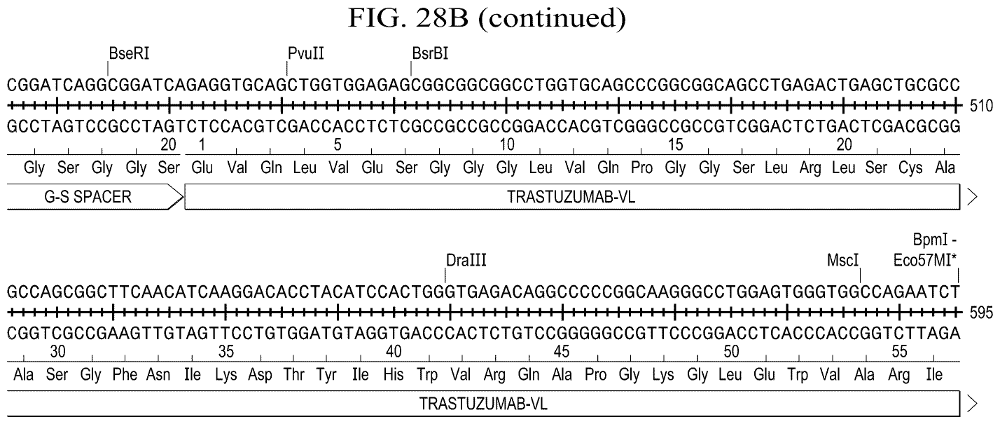

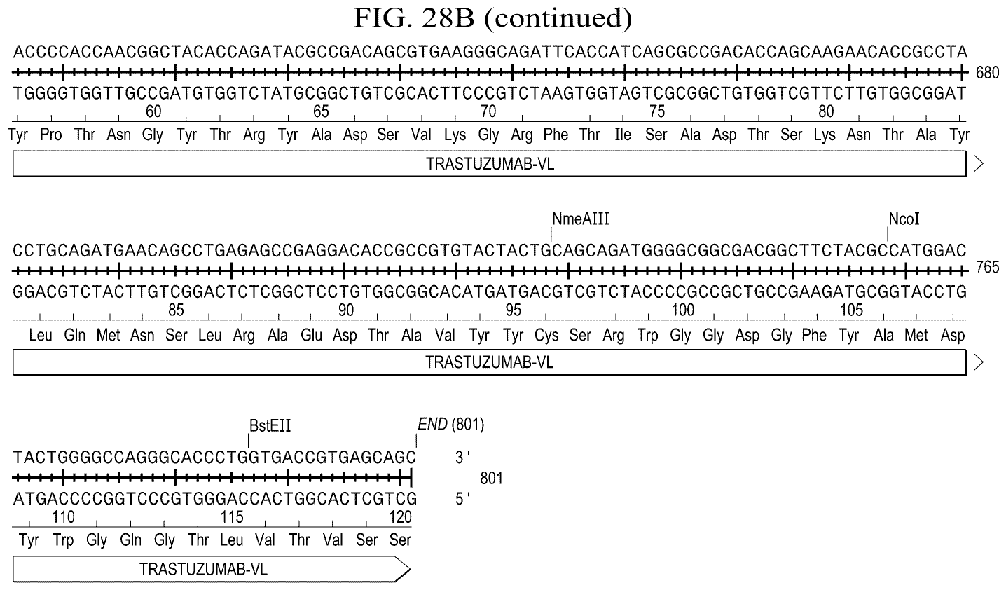

### DETAILED DESCRIPTION

An antigen binding domain (e.g., a single-chain Fv domain (scFv)) may be fused to a CAR scaffold, wherein said scFv gene comprises a VL domain linked to a VH domain via a flexible linker (e.g., SG₂₅ spacer), wherein the CAR scaffold encodes an extracellular domain, a transmembrane domain, and a cytoplasmic domain capable of activation of an immune cell (e.g., such as a lymphocyte cell or a cytotoxic cell). The resultant construct provides antibody or antibody-like specificity to the immune cell due to the antigen binding domain (e.g., an scFv), and the ability to activate the immune cell due to the gene segment comprising the cytoplasmic domain. These techniques may be implemented in T cells, mast cells, and NK cells as well as other suitable cell types.

According to present embodiments, CAR scaffolds and codon-optimized versions of CAR scaffolds are provided for expression of said constructs in immune or other cytotoxic cells, including NK cells, wherein the CAR scaffold is coupled to an antigen binding molecule. In some aspects, these CAR scaffolds are codon optimized for expression in immune or other cytotoxic cells for improved efficacy.

In some aspects, the CAR scaffold may comprise a CD28 domain coupled to a CD32 domain or the complete CD3ζ domain, which is joined to the scFv. For example, the sequence may comprise a leader sequence coupled to an scFv domain coupled to a CAR scaffold, wherein the scFv comprises: VL domain—spacer—VH domain or VH domain—spacer—VL domain.

In some aspects, the spacer may be a SG spacer (e.g., SG25), however, the spacer may comprise any arrangement of amino acids providing sufficient flexibility within the scFv to allow the scFv to bind to its intended target.

In still other embodiments, the leader sequence may be a CD64 leader signal sequence. The CD64 signal sequence may comprise a signal peptide directing transport of the scFv-activation domain to the surface of the cell (e.g., immune or cytotoxic cell).

In some embodiments, the antigen binding molecule may be attached directly or through a linker to the activation region (e.g., this may be accomplished by recombinant expression of a nucleotide encoding the antigen binding molecule, an optional spacer, and a sequence encoding the activation domain). The recombinant expression system may be transfected into a host cell, wherein the fusion protein is expressed, constitutively or inductively.

The antigen binding domain may comprise a leader sequence, a variable region, a linker, and another variable region. The nucleotide sequences encoding these domains may be codon optimized for expression in a particular cell line, leading to higher expression levels of the CAR-based construct and improved efficacy over the non-codon optimized counterparts. It is noted that codon optimization does not alter the polypeptide sequence, but may change codon frequency to optimize expression in host organisms.

Any suitable antigen binding molecule may be attached to the tail activation domain. In some aspects, the antigen binding molecule may comprise a VH and VL domain. The VH and VL domain may be obtained from any suitable monoclonal antibody.

In some aspects, antigen binding molecules may include scFvs, antibodies, aptamers (short sequences of DNA, RNA, or Xeno nucleic acid (XNA) nucleotides), peptides, proteins, protein scaffolds, fusion proteins, or any other suitable molecule that may be engineered to specifically bind to target antigens. In some embodiments, protein scaffolds are related to or derived from peptide aptamers.

In other aspects, the nucleic acid sequence encoding the CD28 domain may comprise a nucleic acid encoding, partially or entirely, the CD28 ectodomain, the CD28 transmembrane domain, and the CD28 cytoplasmic domain.

In still other aspects, CD28 domain may be coupled to the CD3ζ activation domain. The nucleic acid sequence encoding the CD3ζ activation domain may encode at least two ITAMs, at least three ITAMs, etc.

Exemplary sequences are provided as follows. In one aspect, a CAR-19slh2ζ scaffold may comprise a tail end activation domain, such as SEQ ID NO.: 21. This sequence, SEQ ID NO.: 21, comprises various domains, including an ectodomain (encoded by SEQ ID NO.: 27), a transmembrane domain (encoded by SEQ ID NO.: 28), and a cytoplasmic domain (encoded by SEQ ID NO.: 29).

In another aspect, the CAR scaffold contains a CD3 domain that is codon optimized, such as SEQ ID NO.: 23. This sequence, SEQ ID NO.: 23, also comprises various domains, including an ectodomain (encoded by SEQ ID NO.: 24), a transmembrane domain (encoded by SEQ ID NO.: 25), and a cytoplasmic domain (encoded by SEQ ID NO.: 26). Codon optimization may improve expression and thereby improve activity/efficacy of the CAR-based therapeutic composition in its target host cell (e.g., NK cells).

In other aspects, either of the CAR scaffolds (encoded by SEQ ID NO.: 5 or SEQ ID NO.: 22) may be attached to an antigen binding domain comprising a CD64 leader signal sequence (encoded by SEQ ID NO.: 1), a variable heavy (VH) or variable light (VL) chain (see figures for various VH and VL domains), and a GS spacer (encoded by SEQ ID NO.: 3, SEQ ID NO.: 10, SEQ ID NO.: 13, or SEQ ID NO.: 30), wherein the GS spacer joins the VH and VL domains.

In some aspects, sequences having 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over the full sequence or a specified region of the sequence (e.g., nucleotide sequences encoding VH and/or VL domains, CAR scaffold, etc.) are contemplated to fall within the scope of the embodiments provided herein.

Percent identity refers to a specified percentage of amino acid residues or nucleotides that are the same, when compared and aligned for maximum correspondence, over the entire length of the sequence or over a designated region of the sequence. A region may comprise at least 50nucleotides, at least 100 nucleotides, at least 200 nucleotides, or more. A region may comprise at least 10 amino acids, at least 20 amino acids, at least 50 amino acids, or more.

As used herein, “antigen binding domain” refers to a molecule that specifically binds to an antigen on a cell surface (e.g., a malignant or cancer cell). The antigen binding domain includes but is not limited to single-chain Fvs (scFvs) and other antibody fragments as well as affimers, aptamers, peptides, proteins, small molecules, etc. Other antibody fragments include but are not limited to F(ab′)₂, Fab₂, Fab′, Fab, Fv, scFv-Fc, VhH, disulfide-linked Fvs (sdFv), etc. or any active fragment thereof, i.e. antibody fragments or other molecules that immunospecifically bind to an antigen (e.g., EGFR, IGFR-1, FRP5, PD-L1, etc.) or a variant thereof.

It is also understood that any one or more of the CDRs of any antibody fragment may be grafted onto the antigen binding domains described herein. The heavy chains and light chains have a general structure of relatively conserved framework regions (FR) joined by three hyper variable regions or CDRs (CDR1, CDR2, CDR3). The CDRs from the heavy and the light chains, which are aligned by the framework regions, enable binding to the antigen. One of skill in the art would be able to ascertain the CDRs based on known techniques.

The VH and VL domains may be derived from any suitable antibody, including but not limited to monoclonal antibodies, polyclonal antibodies, human antibodies, humanized antibodies, murine antibodies, conjugated antibodies (e.g., to a chemotherapeutic agent, to a radionuclide, to another protein, etc.), synthetic antibodies, bispecific antibodies, chimeric antibodies, single chain antibodies, antibody fragments produced by a Fab expression library, antibody fragments produced by mRNA display or phage display, and monovalent immunoglobulins (e.g., IgG).

Antibodies or fragments thereof may be generated using any suitable technique known in the art, including hybridoma technology, generation of phage displayed scFvs, generation of mRNA displayed scFvs or peptides, or isolation and screening of antisera, chemical synthesis, or through the use of recombinant expression systems.

Antibody fragments (e.g., scFvs) may be screened for binding to a suitable antigen according to techniques known in the art. Affinity maturation may be employed to improve the affinity of a scFv for its desired target. Therapeutic compositions that are administered to a patient include complexes comprising the antibody fragments (e.g., scFvs, etc.) described herein and are usually human or humanized.

Antibody fragments (e.g., scFvs) may be of any origin including but not limited to human, murine (e.g., mouse and rat), donkey, rabbit, goat, guinea pig, bird, camel, horse, or chicken. For therapeutic purposes, antibodies that are human or that have been humanized are preferred.

Any methodology known in the art for screening large combinatorial libraries to identify antibody fragments that bind to an antigen may be applied, including but not limited to phage display, yeast surface display, ribosome display, or mRNA display, or any combination thereof (see, Harlow et al., Antibodies: A Laboratory Manual, Cold Spring Harbor Laboratory Press, 2nd ed. 1988; Hammerling et al., Monoclonal Antibodies and T-Cell Hybridomas (1981) 563-681; WO 98/31700).

As used herein, “specifically binds” typically refers to non-covalent interactions between a target entity (e.g., a cell) and the antigen binding domain, and usually refers to the presence of such an interaction with a particular structural feature (e.g., such as an antigenic determinant on the cell surface) of the target entity with the antigen binding domain. As understood by one of skill in the art, an interaction is considered to be specific if it occurs in the presence of other alternative interactions.

As used herein, “composition” or “pharmaceutical composition” refers to a formulation comprising a therapeutic cell that is delivered to a patient comprising the CAR scaffold coupled to an antigen binding domain and may include one or more additional ingredients (e.g., buffers, excipients, stabilizers, diluents, emulsifiers, preservatives, etc.).

As used herein, and unless the context dictates otherwise, the term “linked to” or “linked with” is intended to include both direct coupling (in which two elements that are coupled to each other contact each other) and indirect coupling (in which at least one additional element is located between the two elements).

In other embodiments, assays may be performed to determine the effectiveness of the compositions. For such detection, an assay that includes the steps of culturing tumor cells under conditions suitable for growth and in a tumor microenvironment, contacting the cells with the CAR-based therapeutic as described herein, and then evaluating whether the CAR-based therapeutic reduces or inhibits the size of the mass may be used.

Components of the compositions disclosed herein can be organized in nearly any manner provided that functional activity for which the complex was designed is maintained. Additionally, the complexes described herein may include one or more tags, e.g., to facilitate modification, identification and/or purification of the components of the complex.

Linkers/Spacers

Linker sequences may be used to link the VH and VL domains of the scFvs, while maintaining desired functional activity of the VH and VL domains. As used herein and unless otherwise noted, the terms “linker sequence,” “linker,” “spacer,” and “G-S spacer” are interchangeable and refer to sequences that connect the VH and VL domains. Where a particular linker sequence is contemplated, it will be associated with a corresponding SEQ ID NO. The linker sequence should allow effective positioning of the VH and VL domains to allow functional activity including binding to their respective antigenic targets.

The linker is preferably encoded by a nucleotide sequence resulting in a peptide that can effectively position the VH and VL domains or other antibody fragment for recognition of its intended target.

In some aspects, the linker sequence is flexible so as to not constrain the VH and VL domains in an undesirable conformation. In some embodiments, the linker comprises amino acids with small side chains, e.g., glycine, alanine and serine, to provide for flexibility. Preferably about 80 or 90 percent or greater of the linker sequence comprises such residues. Exemplary nucleotide sequences encoding linkers are provided in the specification (SEQ ID NO.: 3, SEQ ID NO.: 10, SEQ ID NO.: 13, and SEQ ID NO.: 30), although any suitable linker sequence may be used with the embodiments provided herein. Different linker sequences can be used including any number of flexible linker designs that have been used successfully to join antibody variable regions together, see Whitlow, M. et al., (1991) Methods: A Companion to Methods in Enzymology 2:97-105. Suitable linker sequences can be identified empirically, or determined by computer modeling techniques.

The compositions described herein may be administered in combination with any anti-cancer therapy, including but not limited to, chemotherapeutic agents (such methods are known to the skilled artisan and described in Remington's Pharmaceutical Sciences by E. W. Martin), surgery, radiation therapy, and/or chemotherapy.

### Recombinant Expression Systems

Methods for introducing a polynucleotide into a host cell for expression are well known in the art, and include but are not limited to, integration of the foreign nucleotide sequence into the genome of the cell, vector-based methods wherein the foreign polynucleotide sequence is not integrated into the genome of the cell, and virus-mediated methods.

The sequence of interest (SOI), in this case, the nucleotide sequence encoding the antigen binding domain and the CAR scaffold may be recombinantly expressed in a host cell by inserting the nucleotide sequence into a suitable vector for expression in a mammalian cell and transfecting the vector into the host mammalian cell.

Vectors include DNA molecules into which a genetic insert has been introduced, allowing replication and expression of the insert in a host cell. Vectors include plasmids, viral vectors, cosmids, and artificial chromosomes. Expression vectors typically comprise an origin of replication (ORI), a multicloning site comprising various restriction sites into which an insert can be cloned, and one or more selectable markers (e.g., ampicillin or tetracycline, etc.) for selection. Additionally, the vector can include one or more transcription units, with a transcription unit including a promoter, a polyA signal sequence, and a transcription termination sequence. In some embodiments, the promoter is a mammalian promoter. In other embodiments, the promoter is a viral promoter. In still other embodiments, the promoter is associated with the gene of interest. Promoters can be constitutive or inducible. If induced, a chemical such as IPTG is added to the cell culture in order for the system to recombinantly express the desired protein.

Viral promoters include but are not limited to promoters from adenovirus (such as Adenovirus 2 or 5), cytomegalovirus (CMV), herpes simplex virus (thymidine kinase promoter), retroviral promoters (e.g., MoMLV or RSV LTR), ubiquitin C (UBC), EF1a, PGK, CAGG, and simian virus 40 (SV40). Many other viral promoters are suitable, and all such viral promoters are contemplated herein.

These techniques utilize, unless otherwise indicated, conventional techniques of cell biology, cell culture, molecular biology, virology, microbiology, recombinant DNA, and immunology, all of which are within the skill of an ordinary artisan. Such techniques are explained more fully in the literature. For a description of the functional components of expression vectors, including specific examples of promoters, enhancers, terminal signals, splicing signals, polyA signals, etc., reference is made to the following laboratory manuals that describe standard techniques of molecular biology, and are known by one of skill in the art (see, e.g., Green and Sambrook eds., Molecular Cloning: A Laboratory Manual, 4^{th} edition, Cold Spring Harbor Laboratory, Cold Spring Harbor, N.Y., 2012; Ausubel et al., Current Protocols in Molecular Biology, 3^{rd} ed. 1995; Bothwell et al. Methods for Cloning and Analysis of Eukaryotic Genes, Jones and Bartlett Publ. 1990; Wu, Grossman, Moldave eds. Recombinant DNA Methodology, Academic Press 1989; Adams ed., Cell Culture for Biochemists, Elsevier/North-Holland Biomedical Press, 1990; Butler ed., Mammalian Cell Biotechnology, IRL Press, 1991; Griffiths, et al., recombinant DNA technology in eukaryotes, in An Introduction to Genetic Analysis (2000), New York.

Examples of mammalian expression vectors include but are not limited to adenoviral vectors, adeno-associated vectors, baculovirus vectors, coronavirus, herpes simplex vectors, lentiviruses, pCMV series of plasmid vectors, pSV series of plasmid vectors, retroviral vectors, vaccinia, etc. Various vectors suitable for mammalian expression are derived from viruses, there are many such suitable vectors for expression in mammalian cells and all are contemplated herein.

In embodiments in which integration of the SOI into the host cell genome is desired, lentiviral expression systems may be selected. In embodiments in which integration into the host genome is not desirable, adenovirus expression systems or adeno-associated viruses expression systems may be selected.

Various methods are known in the art for transfection of mammalian expression vectors into host cells for expression, including viral transfection, lipofection, electroporation, calcium phosphate co-precipitation, rubidium chloride or polycation (such as DEAE-dextran)-mediated transfection, protoplast fusion and microinjection, see, e.g., Sambrook et al., for a description of such techniques. Preferably, the transfection method will provide an optimal transfection frequency and expression of the construct in the particular host cell line. Optimization may be performed using well-known techniques in the art.

In some embodiments, bacterial host cells are utilized to propagate mammalian expression vectors for preparation of DNA stocks for subcloning or for introduction into host cells. In other embodiments, bacterial host cells are utilized to produce large quantities of the protein encoded by the SOI (e.g., complexes). Bacterial host cells include but are not limited to E. coli. Yeast host cells include but are not limited to Pichia pastoris.

Suitable expression vectors for bacterial cells include but are not limited to bacterial expression vectors (e.g., E. coli expression vectors such as pGEX and pET series). In still other embodiments, suitable expression vectors include but are not limited to yeast expression vectors (e.g., pPIC series). In still other embodiments, cell-free systems containing the components needed for transcription and translation are provided.

In preferred embodiments, adenoviral expression systems are used. In such systems, an insert comprising a nucleotide encoding the SOI may be cloned into a shuttle vector. The shuttle vector and adenovirus backbone vector (e.g., commercially available systems such as RAPAd® Adenoviral Expression System from Cell Biolabs), are both linearized and are cotransfected into 293 cells to generate a viral stock solution in about 2-3 weeks. The viral stock solution may then be used to transfect target host cells with the nucleotide encoding the SOI(s).

In some embodiments, the transfected cells may be cultured under conditions to express the recombinant protein. In some embodiments, the cells may be subjected to suspension culture. In other embodiments, the cells may be subject to tissue culture. In still other embodiments, expression of the recombinant protein may be induced. In still other embodiments, expression of the recombinant protein may be constitutively expressed.

In some embodiments, the SOI is co-expressed with co-stimulatory molecules, such as cytokines. Immune stimulatory cytokines are added to promote or trigger an immune response. Cytokines include but are not limited to IL2, IL4, IL7, IL11, IL15, IL21, TNF-alpha, IFN-gamma, etc.

### Use with Cytotoxic Cells

Genetically engineered cells may express the constructs provided herein (antigen binding domain coupled to CAR scaffold). These cells can be administered to the patient to recognize and eliminate tumorigenic and/or cancerous cells. Immune response assays may be performed to validate whether or not the engineered cells have activity against the tumor or cancer cell.

NK cells are deemed particularly suitable for use herein, especially where the NK cells are autologous NK cells, obtained from the same individual from which the tumor is obtained.

Alternatively, NK cells may also be grown from monoclonal sources, such as NK-92 or NK-92 derivatives, in which the cells are modified to have a reduced or abolished expression of at least one killer cell immunoglobulin-like receptor (KIR) to render such cells constitutively activated. Such modified cells may be prepared using protocols well known in the art.

Of course, it should also be appreciated that multiple different cell populations may be prepared that have different combinations or sub-combinations of Fc receptors and signaling moieties to so even further increase the anticipated therapeutic effect. For example, two different populations of NK cells may be administered where the first type of Fc receptor is CD16a and where the cell overexpresses Fcγ-signaling subunits, and where the second type of Fc receptor is CD32a and where the cell overexpresses Fcγ-signaling subunits. In another example, two different populations of cells may be administered where the first cell is an NK cell with expressing CD16a and overexpressing Fcγ-signaling subunits, and where the second cell is an CD8+T-cell expressing CD16a and overexpressing Fcγ-signaling subunits. Regardless of the source of the cell, it is generally contemplated that the cell is a mammalian cell, and especially a human cell.

Additionally, and particularly where the cells are not obtained from the mammal that is to receive the subsequently modified cells, it is contemplated that the cells are rendered less immunogenic to the mammal (e.g., via HLA grafting or deletion of MHC complexes).

In yet another example, suitable NK cells for administration may be (or may be derived from) previously established therapeutic cell lines, which are well known in the art. For example, suitable cell lines include aNK cells, haNK cells, taNK cells, NK-92 cells (e.g., commercially available from Nantkwest, 9920 Jefferson Blvd. Culver City, CA 90232) or TALL 104 cells (e.g., commercially available from ATCC, CRL-11386, 10801 University Boulevard, Manassas, VA 20110 USA).

In some cases, the administered cells may be allogeneic, and may be rejected by the recipient's immune system. Thus, in some embodiments, allogeneic cytotoxic cells are modified to be resistant to immunosuppressive agents (e.g., inactivating a gene that is a target for an immunosuppressive agent, e.g., a cyclophilin gene member, a CD52, a FKBP receptor, a glucocorticoid receptor, etc.) so that the cytotoxic cell is capable of functioning in the presence of the immune suppressive agent. The cytotoxic cells are administered in conjunction with immunosuppressive agents including but not limited to an immunosuppressive antimetabolite, a calcineurin inhibitor, a corticosteroid, a dihydrofolic acid reductase inhibitor, an inosine monophosphate dehydrogenase inhibitor, an interleukin-2 ot-chain blocker, or a rapamycin target.

NK cells express a variety of activating receptors, including NKG2D, Ly49 (some are activating, most are inhibitory), KIR (both activating and inhibitory), CD94-NKG2C, and CD94-NKG2E, and inhibitory receptors including Ly49 and KIR. These receptors recognize cellular stress ligands as well as MHC class I and related molecules. (see, Pegram et al., Immunology and Cell Biology (2011) 89:216-224.)

### Immune Activation

Once the CAR-based therapeutic (e.g., an antigen binding domain coupled to a CAR scaffold) binds to the antigen expressed by the cancer cell, the cytotoxic cell can trigger destruction of the cancer cell. While it is generally contemplated that all cytotoxic cells are deemed suitable for use herein, especially preferred cytotoxic cells include NK cells, activated NK cells, high affinity NK cells, CD8+ T-cells, and CD4+ T-cells that have been modified to recombinantly express the CAR-based therapeutic, any of which may be of different origins.

However, it should be appreciated that in other aspects, the cytotoxic cell may also be a macrophage, a monocyte, a neutrophil cell, a basophile, or eosinophil cell. Therefore, and viewed from a different perspective, the cells contemplated herein may effect cytotoxic action via phagocytosis, pore formation, induction of antibody-dependent cell-mediated cytotoxicity (ADCC), by triggering TNF or fas mediated killing pathways, etc.

Cytotoxic cells may release various types of cytotoxic granules (e.g., granulysin, perforin, granzymes) as part of this process. A variety of assays are available for monitoring cell-mediated cytotoxicity, including flow cytometric assays, e.g., based on presence of lytic granules such as perforin, granzymes, or production of TNF family members, e.g., TNF-α, FasL or TRAIL (Zaritskaya 2010, Clay, T. et al., Clin. Cancer Res. (2001) &: 1127-1135).

In one embodiment, a bodily fluid is obtained, wherein the bodily fluid comprises cellular components, e.g., tumorigenic or cancer cells displaying an antigen to which the CAR scaffold expressing cells described herein bind to, and cytotoxic cells expressing the antigen binding moiety are contacted with the cells. Assays are then performed to detect immune responses, e.g., indicating that an ADCC response or an ADCP response has been triggered by the patient's own immune cells.

Assays for detecting an immune response are known in the art and are described herein. For example, assays for detecting such a response may detect a release of cytotoxic granules (e.g., granulysin, perforin, granzymes), or phagocytosis, or receptor-ligand mediated cytolysis (e.g., as mediated by the Fas/APO pathway). A variety of flow cytometric assays are available for monitoring cell-mediated cytotoxicity, e.g., based on presence of lytic granules such as perforin, granzymes, or production of TNF family members, e.g., TNF-α, FasL or TRAIL (Zaritskaya 2010, Clay, T. et al., Clin. Cancer Res. (2001) &:1127-1135).

In other embodiments, immune stimulatory cytokines are administered to a patient in combination with the host cell (expressing a CAR-based therapeutic (e.g., an antigen binding domain coupled to a CAR scaffold)) to promote or trigger an immune response. Cytokines include but are not limited to IL2, IL4, IL7, IL11, IL15, IL21, TNF-alpha, IFN-gamma, etc. In some embodiments, cytokines can reactivate exhausted T cells. In other cases, immune competent cells may be engineered to recombinantly express one or more cytokines.

Techniques to treat cancer include surgery, radiation therapy, chemotherapy, immunosuppressive reagents (e.g., azathioprine, cyclosporin, methotrexate, mycophenolate, etc.), immunotherapy, targeted therapy, hormone therapy, stem cell transplant, or other precision methods. Any of these techniques may be combined with embodiments of the present invention to treat cancer.

It is understood that present invention embodiments may be administered to a patient using appropriate formulations, indications, and dosing regimens suitable by government regulatory authorities such as the Food and Drug Administration (FDA) in the United States.

In some embodiments, a cytotoxic cell expressing a CAR-based therapeutic (e.g., an antigen binding domain coupled to a CAR scaffold) is administered to a patient as a pharmaceutical composition. In another embodiment, a method of treating cancer by administration of the cytotoxic cell to a subject is contemplated. In still another embodiment, a method inhibiting the proliferation or reducing the proliferation of a cell that is expressing the corresponding antigen (to which the antigen binding region binds to) on the surface of its cell by administration of the cytotoxic cell to a subject is contemplated.

In some embodiments, the cytotoxic cell expressing a CAR-based therapeutic (e.g., an antigen binding domain coupled to a CAR scaffold) reduces the amount (e.g., number of cells, size of mass, etc.) by at least 25%, at least 50%, at least 75%, at least 90%, at least 95%, or at least 99% in a subject with cancer associated with expression of the corresponding antigen on the surface of the cells relative to a negative control.

Examples of cancer that are treatable by the cytotoxic cells contemplated herein include any cancer expressing or overexpressing a cancer-associated antigen on its cell surface. Examples of cancer that can be treated with a cytotoxic cell expressing a CAR-based therapeutic (e.g., an antigen binding domain coupled to a CAR scaffold) include but are not limited to breast cancer, colon cancer, leukemia, lung cancer, melanoma, neuroblastoma, pancreatic cancer, pediatric intracranial ependymoma, and prostate cancer.

### Pharmaceutical Compositions

Pharmaceutical compositions may comprise cytotoxic cells comprising an antigen binding domain coupled to or linked to a CAR scaffold, as described herein, in combination with one or more pharmaceutically or physiologically acceptable carriers, diluents or excipients. Additionally, pharmaceutical compositions may comprise one or more adjuvants (e.g., aluminum hydroxide), antioxidants, bacteriostats, buffers, carbohydrates, chelating agents such as EDTA or glutathione; coloring, flavoring and/or aromatic substances, emulsifiers, excipients, lubricants, pH buffering agents, preservatives, salts for influencing osmotic pressure, polypeptides (e.g., glycine), proteins, solubilizers, stabilizers, wetting agents, etc., which do not deleteriously react with the active compounds (e.g., antigen binding domain coupled to a CAR scaffold, etc.) or otherwise interfere with their activity. Buffers include but are not limited to neutral buffered saline, phosphate buffered saline, etc. Carbohydrates include but are not limited to dextrans, glucose, mannose, mannitol, sucrose, etc.

Pharmaceutical compositions may be formulated for a particular mode of administration. Modes of administration may include but are not limited to: intraarticular, intradermal, intranasal, intraperitoneal, intrathecally, intratumoral, intravenous, intraventricularly, subcutaneous, transdermal, transmucosal or topical routes.

In preferred embodiments, the cytotoxic cells are administered by intravenous infusion. Such formulations may be prepared according to standard techniques known by one of ordinary skill in the art. For example, a composition that is to be administered intravenously may have one or more ingredients (e.g., a diluent, a suspension buffer, saline or dextrose/water, other components such as cytokines, etc.) prior to infusion in the patient.

Many such techniques for formulating and administering pharmaceutical compositions are known in the art, e.g., U.S. Patent Application Publication No. 2014/0242025, and all such references are incorporated by reference herein in their entirety.

In some embodiments, the cytotoxic cells proliferate in vivo, thereby persisting in the patient for months or even years after administration to provide a sustained mechanism for inhibiting tumor growth or recurrence. In some aspects, the cytotoxic cells persist at least for three months, six months, nine months, twelve months, fifteen months, eighteen months, two years, three years, four years, or five years after administration of the cytotoxic cells to the patient.

Cytotoxic cells may be obtained from any of a variety of sources, (e.g., isolated from a human, from commercially available cytotoxic cells, from a cell repository, etc.). Procedures for ex vivo expansion of NK cells, T cells or other types of cytotoxic cells are known in the art (e.g., Smith et al., Clinical & Translational Immunology (2015) 4: e31). The examples presented herein are not intended to be limited to any particular method of ex vivo expansion of cytotoxic cells.

Pharmaceutical compositions comprising cytotoxic cells, as described herein, may be administered at a dosage of 10⁴ to 10⁹ cells/kg body weight, of 10⁵ to 10⁶ cells/kg body weight, or any integer values within these ranges. Cytotoxic cell compositions may be administered one time or serially (over the course of days or weeks or months) at these dosages. Infusion techniques for cytotoxic cells, such as T cells, are known in the art (see, e.g., Rosenberg et al., New Eng. J. of Med. 319:1676, 1988).

In other embodiments, the pharmaceutical compositions are administered in a therapeutically effective amount, which is the amount effective for treating the specific indication. Administration may occur as a one-time dose or based on an interval. As used herein, “interval” indicates that the therapeutically effective amount is administered periodically (as distinguished from a one-time dose). The administration interval for a single individual need not occur at a fixed interval, but can vary over time. The term, “in combination with” or “co-administered” indicates that a composition can be administered shortly before, at or about the same time, or shortly after another composition.

It should be apparent to those skilled in the art that many more modifications besides those already described are possible without departing from the concepts herein. The present subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C and N, the text should be interpreted as requiring only one element from the group, not A plus N, or B plus N, etc.

### EXAMPLES

### Example 1. Generation of Antigen Binding Domain-CAR Scaffold

A variety of techniques are available for generation of the constructs provided herein. These constructs comprise an antibody fragment (e.g., a scFv, one or more CDRs, etc.) fused to a linker, which is fused to a CAR scaffold. Various protocols for generating these constructs are known to one of ordinary skill in the art.

In one embodiment, the nucleotide sequence encoding for a scFv may be obtained according to the techniques presented herein (e.g., phage display, mRNA display, from monoclonal antibodies, etc.). In some cases, the VH and VL domains may be known in the art, and linked together using known techniques to form an scFv. The nucleotide sequence corresponding to the scFv may be coupled to a CAR scaffold. Once the full nucleotide sequence is obtained, it may ligated into a suitable vector. Immune cells are then transfected with the vector, the construct is expressed, and the engineered cells are subjected to the assays disclosed herein.

CAR constructs direct NK cells to a particular target as NK cells without relying on HLA matching (unlike T cells) (see, e.g., Hermanson et al., Front Immunol (2015) 6:195; and Carlsten et al., Front Immunol (2015) 6:266). Various NK cell lines contemplated herein include but are not limited to aNK, HaNK, NK-92, NKG, NKL, NK-YS, TaNK, YT, and YTS cells.

Also contemplated herein is transfection with genes encoding for one or more cytokines, e.g., IL2, IL4, IL7, IL11, IL15, IL21, TNF-alpha, IFN-gamma. In some embodiments, IL-2 and/or IL-15 are transfected to promote in vivo expansion and persistence.

Cytotoxic cells may be genetically modified (i.e., transduced or transfected in vitro) with a vector encoding the constructs disclosed herein. The cytotoxic cell may be administered to a mammalian recipient to provide a therapeutic benefit, namely directing cytotoxic cells to destroy cells expressing the antigen that the antigen binding moiety binds to on their surface, e.g., cancer cells.

The mammalian recipient may be a human and the cytotoxic cell can be autologous with respect to the recipient. Alternatively, the cells can be allogeneic, syngeneic or xenogeneic with respect to the recipient. In some embodiments, allogeneic cells are administered with an immune suppressant.

### Example 2. Cell Killing Assays

Killings assays are known in the art, e.g., at U.S. Pat. No. 7,741,465. For example, as provided in the '465 patent, the ability of the transfected cells to mediate specific target cell killing was determined by a ⁵¹Cr release assay.

For example, engineered cells may be transformed with constructs provided herein, and a level of cell lysis measured, based on a ⁵¹Cr release signal when compared to an untransfected control. Transfected cells will generally induce a significantly higher specific ⁵¹Cr release from human tumors or cancer cells than the corresponding control cells.

### Example 3. Detection of an Immune Response

The ability of genetically engineered cells to elicit an immune response may be tested. A variety of assays for monitoring cellular immune responses in vivo and in vitro are available (see, e.g., Clay T. et al., Clin. Cancer Res. (2001) p1127-1135).

In some embodiments, cytotoxic cells expressing the constructs provided herein (antigen binding domain coupled to a CAR scaffold) can be tested to determine whether an immune response is triggered, e.g., by detecting a release of cytotoxic granules, phagocytosis, or receptor-ligand mediated cytolysis, from lysed tumor cells.

Traditional assays for measuring cell lysis include addition of a radioisotope, e.g., ⁵¹Cr, to cell culture, which is trapped in the interior of living cells. The radioisotope is released upon cell lysis into the extracellular fluid, providing an indicator of the amount of lysis occurring.

Other assays exist in which levels of Granzyme B are measured. Granzyme B is secreted by activated cytotoxic T cells or NK cells. Granzyme B is released through exocytosis, and in conjunction with perforin, is able to enter target cells to help trigger cell death. Enzyme linked immunoassays (e.g., ELISpot, an ELISA sandwich assay) are known in the art for quantifying the amount of secreted Granzyme B. Essentially, cells are incubated in the presence of antibodies specific for Granzyme B. The cells are removed, and a second Granzyme B specific antibody is added with a detectable marker (e.g., biotin/alkaline phosphatase streptavidin complex). Based on the intensity of color formation, the amount of Granzyme B can be quantified (see, www.mdsystems.com/products/human-granzyme-b-elispot-kit_el2906#product-details; Malyguine A. ct al., Cells (2012) 1(2): 111-126).

1-20. (canceled)

21. An isolated nucleic acid sequence, optimized for expression in a mammalian cell, encoding a chimeric antigen receptor (CAR) polypeptide, wherein the CAR polypeptide comprises an antigen binding domain coupled to a CAR scaffold, wherein the antigen binding domain comprises a single chain Fv (scFv) domain that binds a tumor antigen.

22. The isolated nucleic acid of claim 21, wherein the scFv domain binds a tumor antigen comprising CD19, EGFR, IGFR-1, FRP5, or PD-L1.

23. The isolated nucleic acid of claim 22, wherein the scFV domain binds CD19.

24. The isolated nucleic acid of claim 23, wherein the scFv domain comprises an amino acid sequence according to SEQ ID NO: 6 or an amino acid sequence with 85% or greater identity to SEQ ID NO: 6.

25. The isolated nucleic acid sequence of claim 21, wherein the nucleic acid sequence encoding the scFv domain is human, humanized, synthetic or chimeric.

26. The isolated nucleic acid sequence of claim 21, wherein the scFv domain comprises a linker, and wherein the linker comprises glycine and serine amino acids.

27. The isolated nucleic acid sequence of claim 26, wherein the linker comprises about 15 to 25 amino acids.

28. The isolated nucleic acid sequence of claim 27, wherein the linker is encoded by a nucleic acid sequence according to SEQ ID NO: 3, SEQ ID NO: 10, SEQ ID NO: 13, SEQ ID NO: 30, or a nucleic acid sequence with 85% or greater identity to SEQ ID NO: 3, SEQ ID NO: 10, SEQ I DNO: 13 or SEQ ID NO: 30.

29. The isolated nucleic acid of claim 21, wherein the antigen binding domain further comprises a CD64 leader signal sequence.

30. The isolated nucleic acid of claim 21, wherein the CAR scaffold comprises a CD28 domain coupled to a CD34 domain.

31. The isolated nucleic acid of claim 30, wherein the CD28 domain comprises:
a CD28 ectodomain encoded by a nucleic acid sequence according to SEQ ID NO: 27 or a nucleic acid sequence with 85% identity to SEQ ID NO: 27;
a CD28 transmembrane domain encoded by a nucleic acid sequence according to SEQ ID NO: 28 or a nucleic acid sequence with 85% identity to SEQ ID NO: 28; and
a CD28 cytoplasmic domain encoded by a nucleic acid sequence according to SEQ ID NO: 29 or a nucleic acid sequence with 85% identity to SEQ ID NO: 29.

32. The isolated nucleic acid of claim 30, wherein the CD33 domain comprises a CD33 domain comprising at least two immunoreceptor tyrosine-based activation motifs (ITAMs).

33. The isolated nucleic acid of claim 32, the CD3ζ domain comprises:
a CD3ζ ectodomain encoded by a nucleic acid sequence according to SEQ ID NO: 24 or a nucleic acid sequence with 85% identity to SEQ ID NO: 24;
a CD3ζ transmembrane domain encoded by a nucleic acid sequence according to SEQ ID NO: 25 or a nucleic acid sequence with 85% identity to SEQ ID NO: 25; and
a CD3ζ cytoplasmic domain encoded by a nucleic acid sequence according to SEQ ID NO: 26 or a nucleic acid sequence with 85% identity to SEQ ID NO: 26.

34. The isolated nucleic acid of claim 30, wherein the CAR scaffold is encoded by a nucleic acid sequence according to SEQ ID NO: 5 or a nucleic acid sequence with 85% or greater identity to SEQ ID NO: 5.

35. A vector comprising the isolated nucleic acid sequence of claim 21.

36. An immune cell comprising the vector of claim 35.

37. An immune cell comprising the isolated nucleic acid sequence of claim 21.

38. An NK cell comprising the isolated nucleic acid sequence of claim 21.

39. An NK cell comprising the vector of claim 35.

40. A method for making an immune cell comprising transducing or transfecting the immune cell with the isolated nucleic acid of claim 21.

41. A method for treating a cancer in a subject in need thereof, the method comprising administering an effective amount of an immune cell expressing the isolated nucleic acid encoding the chimeric antigen receptor (CAR) polypeptide of claim 21 to the subject, thereby causing selective depletion of cancer cells.

42. A chimeric antigen receptor (CAR) polypeptide comprising:
a signal sequence;
an antigen binding domain comprising a single chain Fv (scFv) domain that binds a tumor antigen; and
a CAR scaffold.

43. The CAR polypeptide of claim 42, wherein the scFv domain binds a tumor antigen comprising CD19, EGFR, IGFR-1, FRP5, or PD-L1.

44. The CAR polypeptide of claim 43, wherein the scFV domain binds CD19.

45. The CAR polypeptide of claim 44, wherein the scFv domain comprises SEQ ID NO: 6 or an amino acid sequence with 85% or greater identity to SEQ ID NO: 6.
